(19) United States
(12) Patent Application Publication
(10) Pub. No.: US 2026/0148849 A1
(43) Pub. Date: May 28, 2026

(54) DETERMINING TUMOR HETEROGENEITY BASED ON FRAGMENTOMIC FEATURES

(71) Applicant: Foundation Medicine, Inc., Cambridge, MA (US)

(72) Inventors: Ethan S. Sokol, Somerville, MA (US); Zoe R. Fleischmann, Jamaica Plain, MA (US)

(21) Appl. No.: 19/252,701

(22) Filed: Jun. 27, 2025

Related U.S. Application Data
(60) Provisional application No. 63/723,962, filed on Nov. 22, 2024.

(51) Int. Cl. G16H 50/20; C12Q 1/6855; C12Q 1/6874; G16B 20/20; G16B 40/20; G16H 15/00; G16H 20/10; G16H 50/30; G16H 50/70

(52) U.S. Cl. CPC G16H 50/20; C12Q 1/6855; C12Q 1/6874; G16B 20/20; G16B 40/20; G16H 15/00; G16H 20/10; G16H 50/30; G16H 50/70; C12Q 2600/106

(57) ABSTRACT

Techniques for identifying a tumor heterogeneity of a subject are described. In an example method, sequence read data of a sample obtained from the subject is identified. The sequence read data is indicative of endpoint positions of nucleic acid molecules in the sample. The example method further comprises determining endpoint positions of the nucleic acid molecules, generating input features based on the endpoint positions of the nucleic acid molecules, and classifying, using a classifier, the tumor heterogeneity of the subject based on the input features.

### CROSS-REFERENCE TO RELATED APPLICATION

This application claims priority to U.S. Provisional Application No. 63/723,962, which was filed on Nov. 22, 2024 and is incorporated by reference herein in its entirety.

### BACKGROUND

Many individuals rely on genetic testing to identify whether they have, or are predicted to develop, various health related conditions. In some cases, single gene testing can be used to assess whether an individual has a particular genetic mutation that is relevant to whether the individual has a genetic disorder or a propensity for disease. Multiple genes, in some cases, can be tested in order to provide even greater context into the individual's health. Whole exome sequencing (WES) and whole genome sequencing (WGS) can provide even further context.

Extensive genomic sequencing methodologies, such as those utilizing sequence read data obtained by WGS, can result in a substantial amount of data for analysis. It may be difficult to process this substantial amount of data, directly, to accurately identify whether an individual has a particular condition, such as a type of cancer. For instance, a substantial amount of processing resources may be utilized in order to identify a condition of a subject using sequence read data. Moreover, some conditions are not apparent by evaluating sequence read data directly.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 illustrates an example environment for predicting a tumor heterogeneity of a subject based on fragmentomic features of the subject.

FIG. 2 illustrates example signaling for selecting features for classifying a tumor heterogeneity of a subject based on fragmentomic data of the subject.

FIG. 3 illustrates example signaling for preprocessing fragmentomic data for use in classification.

FIG. 4 illustrates an example environment for training and utilizing a predictive model to identify a tumor heterogeneity of a subject.

FIG. 5 illustrates an example of training data utilized to train one or more machine learning (ML) models to identify a tumor heterogeneity of a subject.

FIG. 6 illustrates an example report summarizing the predicted conditions (e.g., a tumor heterogeneity) of a subject.

FIG. 7 illustrates an example environment for sequencing various nucleic acid molecules, such as nucleic acid fragments.

FIG. 8 illustrates an example environment illustrating cell-free DNA (cfDNA), which can be utilized to identify heterogeneity of a tumor of a subject.

FIG. 9 illustrates an example process for identifying a heterogeneity condition of a subject using transformed data.

FIG. 10 illustrates one or more devices configured to perform various operations described herein.

### DETAILED DESCRIPTION

Various implementations of the present disclosure relate to techniques for predicting health-related conditions, such as a tumor heterogeneity, based on nucleic acid sequencing data. In various cases, nucleic acid molecules are obtained from a subject. In some cases, the nucleic acid molecules include DNA fragments (e.g., cfDNA) obtained from a liquid biopsy sample. Sequence read data is generated by sequencing the nucleic acid molecules. In various cases, the sequence read data includes at least one dimension that represents a position of the sequenced nucleic acid molecules in a reference genome (also referred to as a “genomic position”), such that the sequence read data is in a spatial domain.

In some aspects, the sequence read data is preprocessed. In some examples, the sequence read data is preprocessed in the spatial domain. According to some examples, the sequence read data is normalized and/or smoothed. In various implementations of the present disclosure, the sequence read data is transformed into an alternate domain, before or after preprocessing. For instance, the sequence read data may be transformed into a frequency or wavelet domain by performing an appropriate transform on the sequence read data. The transformed sequence read data (also referred to as “transformed data”) exhibits various features of the subject that are difficult to impossible to ascertain in the original domain of the sequence read data. These features, for instance, are predictive of a tumor heterogeneity. According to various examples, the features of the transformed data are used to determine the tumor heterogeneity of the subject. For instance, the features may be input into a predictive model that is configured to determine whether the subject has the tumor heterogeneity. In various cases, indications of the tumor heterogeneity of the subject are reported to the subject directly or to a care provider that is responsible for the subject.

Various types of health-related conditions can be predicted using various techniques described herein. In some cases, these techniques are used to determine whether the subject has a heterogeneous tumor or a particular tumor heterogeneity. For instance, these techniques can be used to determine a number, a proportion, or one or more cell types of cell populations in a tumor of the subject. In various cases, these techniques can be used to determine a metric indicative of a genomic variation (e.g., genetic variation) and/or a phenotypic variation associated with the tumor of the subject. These techniques can be used, in various cases, to determine a tumor evolution of the subject, a predicted tumor evolution of the subject, a predicted tumor progression of the subject, a predicted metastasis profile of the subject, or a prognosis (e.g., a predicted survivability) of the subject. In some cases, these techniques can be used to determine one or more therapeutic agents predicted to treat the cell populations in the tumor of the subject.

Implementations of the present disclosure provide significant improvements to the technical field of medical diagnostics and treatment. Utilizing sequence read data of DNA fragments and/or the preprocessing techniques described herein may greatly enhance the accuracy of predictions of tumor heterogeneity. In some cases, the techniques described herein can be used to predict whether a subject has a particular condition with high (e.g., 90%, 95%, 99%, or the like) accuracy using nucleic acid molecules that are obtained using a minimally invasive liquid biopsy process. Accordingly, the subject and care providers may make informed decisions about the subject's health without the subject being subjected to highly invasive procedures, such as surgeries (e.g., tissue biopsy procedures). In some examples, the sequence read data and/or the preprocessing techniques described herein may identify new tumor heterogeneity conditions and/or new therapeutics (e.g., new targets for treatment, new indications for existing therapeutics, new combination treatments, etc.) for treating tumors with particular heterogeneity conditions.

Various analyses described herein cannot be performed in the human mind, or by pen and paper. For example, it would not be possible to preprocess or transform sequence read data representing numerous (e.g., hundreds, thousands, etc.) of bases in a sample into an alternate domain (e.g., a frequency domain) solely in the mind of a human. In addition, it would be impossible to manually or mentally identify relevant features based on the preprocessed sequence read data. Particular implementations of the present disclosure are fundamentally tied to computer technology, and do not represent mere automation of processes that are performed manually or within the human mind.

Implementations of the present disclosure utilize a unique and inventive sample type for predicting occurrence of tumor heterogeneity. Previously, tumor heterogeneity was identified using histopathological examination of excised tissue or using sequencing-based approaches. Examples of previously used sequencing-based approaches include the detection of specific genomic variants, which may be limited to known regions of interest, and whole genome approaches, which can be limited by resolution and/or depth, using excised tissue. In contrast, the present disclosure describes implementations of predicting tumor heterogeneity using nucleic acid fragments, such as DNA fragments present in blood, plasma, or some other sample type that can be obtained using a minimally invasive procedure. Further, the present disclosure describes implementations of identifying regions of interest associated with tumor heterogeneity, rather than relying solely on known regions of interest. Further, in various implementations described herein, occurrence of tumor heterogeneity can be predicted as part of a screening procedure, such as before symptoms develop.

### Example Definitions

The terms “deoxyribonucleic acid,” “DNA,” “DNA molecule,” and their equivalents, may refer to a polymer of nucleotides (also referred to as “nucleobases”) containing deoxyribose. The nucleotides in DNA include cytosine (C), guanine (G), adenine (A), and thymine (T). Each DNA nucleotide includes a deoxyribose and a phosphate group. An example single-stranded DNA (ssDNA) molecule includes a chain of covalently bonded DNA nucleotides. In the example ssDNA molecule, the phosphate group of the mth nucleotide is covalently bonded to the deoxyribose of the (m-1)th nucleotide, wherein m is a positive integer greater than 2 and less than or equal to the number of DNA nucleotides in the chain. In various examples, DNA is double-stranded and includes two ssDNA molecules that are complementary to one another and coiled around each other in a double helix form. The nucleotides of one ssDNA molecule are hydrogen bonded to the nucleotides of the other ssDNA molecule. In particular, the pyrimidines (A and T) hydrogen bond to each other, and the purines (C and G) hydrogen bond to each other.

The terms “ribonucleic acid,” “RNA,” “RNA molecule,” and their equivalents, may refer to a polymer of nucleotides containing ribose. The nucleotides in RNA include cytosine (C), guanine (G), adenine (A), and uracil (U). Each RNA nucleotide includes a ribose and a phosphate group. In an example RNA molecule, the phosphate group of the nth nucleotide is covalently bonded to the ribose of the (n-1)th nucleotide, wherein n is a positive integer greater than 2 and less than or equal to the number of RNA nucleotides in the chain. Messenger RNA (mRNA) is a type of RNA molecule that is synthesized (or “transcribed”) by RNA polymerase (an enzyme) to be complementary to a gene encoded in a DNA sequence, and is also used by a ribosome to synthesize a polypeptide or protein. An mRNA is therefore an example of a “coding RNA.” In various cases, intron sequences are removed from an mRNA via a process known as “RNA splicing.” MicroRNA (“miRNA”) are single-stranded RNA molecules that perform post-transcriptional gene expression regulation. For instance, a miRNA may bind to a complementary mRNA molecule, thereby cleaving, destabilizing, or otherwise preventing the mRNA molecule from being translated into a polypeptide or protein by a ribosome. In various examples, a miRNA has a length in a range of 21 to 23 RNA nucleotides. As used herein, the terms “non-coding RNA” may refer to a type of RNA that is not translated into a protein. Examples of non-coding RNA include miRNA, transfer RNA (tRNA), ribosomal RNA (rRNA), small interfering RNA (siRNA), Piwi-interacting RNA (piRNA), small Cajal body-specific RNA (scaRNA), long intergenic non-coding RNA (lincRNA), circular RNA (circRNA), enhancer RNA (eRNA), or natural antisense transcripts (NAT). The term “functional RNA,” and its equivalents, may refer to any RNA molecule that impacts a biological process. For instance, functional RNA may include mRNA, miRNA, tRNA, rRNA, and the like.

The term “base,” and its equivalents, may refer to a monomer of a polymer. For example, a base of DNA or RNA is a nucleotide.

The term “base pair,” and its equivalents, may refer to a pair of complementary DNA nucleotides, which are hydrogen-bonded to one another in a double-stranded DNA molecule. For example, a base pair includes a first base in a first ssDNA and a second base in a second ssDNA, wherein the first and second bases are complementary and hydrogen-bonded to one another.

The terms “nucleotide,” “nucleobase,” “nucleic acid,” “nucleic acid molecule,” and their equivalents, may refer to an organic molecule that includes a nitrogenous base, a sugar, and a phosphate group. In various cases, a nucleotide is a monomer of DNA or RNA. A nucleotide, for instance, is a chemical structure.

The terms “3′ end,” “3-prime end,” and their equivalents, may refer to a terminus of a single-stranded nucleotide polymer that includes a base whose third carbon in its deoxyribose or ribose is bound to a hydroxyl group while being unbound to another base.

The terms “5′ end,” “5-prime end,” and their equivalents, may refer to a terminus of a single-stranded nucleotide polymer that includes a base whose fifth carbon in its deoxyribose or ribose ring is unbound to another base. In some cases, the fifth carbon is bound to a phosphate group.

The “length” of a polymer refers to a number of covalently bonded monomers that are included in the polymer. For instance, the length of a DNA molecule may be the number of covalently bonded nucleotides in at least one strand of the DNA molecule and/or the number of base pairs in the DNA molecule. In various examples, the length of an RNA molecule may be the number of covalently bonded nucleotides in the RNA molecule.

The term “gene,” and its equivalents, refers to a sequence of DNA nucleotides that is transcribed into a functional RNA. The functional RNA, for instance, is RNA that is translated into a polypeptide or protein (e.g., mRNA) or that has some other biological function (e.g., miRNA, tRNA, etc.). A gene is “expressed” when it is used as a template to generate a functional RNA. A subject, for instance, has numerous genes contained in the subject's genome. A gene may include both introns and exons. As used herein, the term “intron,” and its equivalents, may refer to a subset of DNA nucleotides in a gene that is not used to code for any functional RNA that is expressed by the organism. As used herein, the term “exon,” and its equivalents, may refer to a subset of DNA nucleotides in a gene that is used to code for a functional RNA. For instance, an exon may encode a polypeptide or protein that is expressed by the organism. In various examples, a gene can be represented in data (e.g., as data representative of the sequence of DNA nucleotides in the gene) or as a chemical structure (e.g., as the sequence of DNA nucleotides itself).

The term “genome,” and its equivalents, refers to the aggregate of genes of a subject (and optionally non-coding regions). In various cases, a genome represents the sequences of several linear DNA molecules that are present in a subject's chromosomes. A “reference genome” refers to an aggregation of genes of one or more reference subjects. In various cases, a genome is represented in data.

The terms “pangenome,” “pan-genome,” “supragenome,” and their equivalents, refers to an aggregate set of genes from multiple subgroups (e.g., strains) within a population (e.g., a clade) of subjects. A pangenome, for example, indicates genes that are present in all subjects within the population, as well as genes that are present in some of the subjects of the population. A pangenome is represented in data, for instance.

The term “transcriptome,” and its equivalents, refers to the aggregate of RNA sequences of a subject. In some cases, a transcriptome is limited to mRNA sequences. In various examples, a transcriptome is represented in data.

The terms “genomic DNA,” “gDNA,” “chromosomal DNA,” and their equivalents, may refer to DNA molecules that are obtained from a chromosome and/or nucleus of a cell.

The terms “DNA fragment,” “fragment,” and their equivalents, may refer to DNA molecules that are excised and/or broken off from a larger DNA molecule.

The terms “cell-free DNA,” “cfDNA,” and their equivalents, may refer to DNA fragments that are non-encapsulated and obtained outside of cells within a sample (e.g., a liquid biopsy sample).

The terms “circulating tumor DNA,” “ctDNA,” and their equivalents, may refer to a cfDNA molecule that originates from a cancer cell.

The terms “end motif,” “terminal sequences,” and their equivalents, may refer to a sequence of nucleotides extending from a 3′ or 5′ end of a DNA or RNA molecule. In various cases, the end motif is shorter than a length of the DNA or RNA molecule. For example, the end motif may have a length in a range of 5 to 30 bases or base pairs, a range of 3 to 30 bases or base pairs, or a range of 1 to 30 base pairs.

The term “promoter,” and its equivalents, may refer to a portion of a DNA molecule that binds one or more proteins in order to initiate transcription of a gene. For example, the promotor is located “upstream” of the gene. For example, the promotor is located between the 5′ end of the DNA molecule and the gene. A promotor may include one or more binding sites for RNA polymerase, and/or one or more transcription factor binding sites. In some examples, a promotor includes one or more CpG islands. A promoter, for instance, includes a transcription start site.

The terms “CpG island,” “CGI,” “CpG site,” and their equivalents, may refer to a continuous portion of a DNA molecule whose sequence includes greater than a threshold amount (e.g., greater than 50%) of G-C base pairs.

The term “DNA methylation test” and its equivalents may refer to an assay, which can be commercially available, for distinguishing methylated versus unmethylated cytosine loci in DNA. Techniques for measuring cytosine methylation include bisulfite-based methylation assays. The addition of bisulfite to DNA results in the methylation of unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine. Enzymatic methylation sequencing is an example of a DNA methylation test.

An exemplary quantitative methylation detection assay combines bisulfite treatment and restriction analysis COBRA, which uses methylation sensitive restriction endonucleases, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay can be performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight™ (Qiagen, Redwood City, CA) a quantitative methylation detection assay that uses fluorescence-based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are then used to amplify the target sequence of interest. The amplified PCR product is isolated and used to quantitate the methylation status of the CpG site of interest. (Gonzalgo and Jones Nuclei Acids Res. 1997; 25:252-31).

In particular embodiments, pyrosequencing can be used to detect marker methylation. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, converting unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al. Methods Mol Biol. 2015 1343:249-264. Pyrosequencing is especially useful for detecting methylation in the CpG sites within genes.

In particular embodiments, a protein marker is detected by contacting a sample with reagents (e.g., antibodies), generating complexes of reagent and marker(s), and detecting the complexes. Particular embodiments for detecting and measuring protein levels can use methods including agglutination, chemiluminescence, electro-chemiluminescence (ECL), enzyme-linked immunoassays (ELISA), immunoassay, immunoblotting, immunodiffusion, immunoelectrophoresis, immunofluorescence, immunohistochemistry, immunoprecipitation, mass-spectrometry, and western blot. See also, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

The term “enhancer,” and its equivalents, may refer to a portion of a DNA molecule that binds one or more proteins (or regulatory RNA) in order to increase the chance that a gene will be transcribed. For instance, an enhancer includes one or more transcription factor binding sites. In various cases, an enhancer includes one or more CpG islands.

The term “condition,” and its equivalents, may refer to the state of an individual's health. A condition may refer to a positive state (e.g., a visual acuity that is better than 20/20 vision, nonpathological hypotension, etc.), a normal state (e.g., a normal blood pressure), a negative state (e.g., a pathological condition, such as cancer), or any combination thereof.

The term “pathological condition,” “pathology,” “disease,” and their equivalents, may refer to an abnormal anatomical, physiological, or psychological condition that reduces one or more functional abilities below a typical efficiency. As a result of a pathological condition, a subject may have an impaired function, pain, reduced life expectancy, or some other negative health consequence.

The term “cancer,” and its equivalents, may refer to a condition of a subject in which particular cells (referred to as “cancer cells”) divide uncontrollably in the subject's body. In some cases, a cancer is characterized by a location or tissue type from which the cancer cells originated. In some examples, a cancer is characterized by a location or tissue type in which the cancer cells are located. Cancer is a type of pathological condition.

The terms “tumor,” “neoplasm,” and their equivalents, may refer to a mass of tissue including cancer cells.

The term “primary tumor,” and its equivalents, may refer to an original tumor that has grown at the initial site of cancer progression. The anatomical location of the primary tumor may be referred to as a “primary site.”

The term “secondary tumor,” and its equivalents, may refer to a malignant tumor that has spread from the primary site. A secondary tumor, for example, includes the same type of cancer cells as the primary tumor, but the secondary tumor is located in a different anatomical location than the primary tumor.

The term “tumor heterogeneity,” “heterogeneity,” and their equivalents, may refer to variations in genomic DNA (e.g., genomic heterogeneity and/or genetic heterogeneity) and/or variations in physical characteristics (e.g., phenotypic heterogeneity) of cells within a tumor. Tumor heterogeneity, for example, may refer to the presence of distinct cell populations within a tumor. A population of cells may be characterized by the genomic DNA and/or the physical characteristics of the cells. In some examples, tumor heterogeneity may refer to the one or more cell populations within a particular tumor of a subject. In some examples, a “heterogeneous tumor” may refer to a tumor with more than one cell population. A “homogenous tumor,” in various examples, may refer to a tumor with one cell population.

The term “tumor evolution,” “evolution,” and their equivalents, may refer to genomic changes in cancer cells within a tumor. In some examples, tumor evolution may be represented by an evolutionary tree of the tumor. Tumor evolution, for instance, may indicate a clonal evolution of the tumor. The clonal evolution may indicate when a given clone originated and/or one or more mutations associated with the given clone.

The terms “circulating tumor cells,” “CTCs,” and their equivalents, may refer to cancer cells that have separated from a tumor and have entered the bloodstream.

The terms “tissue of origin,” “tissue origin,” and their equivalents, may refer to a differentiated type of tissue from which cancer cells in the body of a subject began dividing uncontrollably in the subject's body.

The terms “liquid biopsy,” “fluid biopsy,” and their equivalents, may refer to a process of obtaining a fluid sample from a subject's body. The sample, for instance, can be referred to as a “liquid biopsy sample.” Examples of fluids that are sampled from the body include blood, plasma, cerebrospinal fluid, sputum, stool, urine, lymphatic fluid, and saliva.

The term “tissue biopsy,” and its equivalents, may refer to a process of obtaining a sample of cells from a subject's body. A tissue biopsy, in various cases, is performed by cutting a mass of cells from the subject's body. For instance, a tissue biopsy is a procedure performed by a surgeon, interventional radiologist, interventional cardiologist, or other specialized clinician. The term “tissue” or “tissue biopsy sample” can be used to refer to the sample of cells obtained using a tissue biopsy.

The term “viral status test” and its equivalents may refer to a test that identifies the presence of viral RNA or DNA in a subject. The test can identify viral load and/or viral identity. For example, the viral status test can identify the presence of viral RNA or DNA associated with the occurrence of certain cancers and non-cancer conditions. Examples of such viruses include Hepatitis B Virus (HBV) and Hepatitis C Virus (HCV), Kaposi Sarcoma-Associated Herpesvirus (KSHV), Merkel Cell Polyomavirus (MCV), Human Papillomavirus (HPV), Human Immunodeficiency Virus Type 1 (HIV-1, or HIV), Human T-Cell Lymphotropic Virus Type 1 (HTLV-1), and Epstein-Barr Virus (EBV).

The term “subject,” and its equivalents, may refer to a human or non-human animal. A subject that is receiving care from at least one care provider may be referred to as a “patient.”

The term “variant,” and its equivalents, may refer to a difference between a subject's genetic sequence and a reference sequence. For instance, a variant may correspond to a difference between one or more nucleotides in a genome of a subject and one or more corresponding nucleotides in at least one reference genome or pangenome. A variant may be characterized by its identity (e.g., what nucleotides are different), its position (e.g., where are the nucleotides located in the genome, what chromosome contains the nucleotides, what gene contains the nucleotides, etc.), its length (e.g., how many nucleotides are different from the reference sequence), its type (e.g., substitution, insertion, deletion, copy number alternation, rearrangement of fusion, etc.), and other features that indicates its significance and/or relevance. In some cases, a variant represents any apparent alteration in a sequence that has been read from a nucleic acid molecule with respect to the reference sequence, such as reads cleaved by restriction enzymes (RE). In various examples, a variant can be represented in data (e.g., by data characterizing the variant) or as a chemical structure (e.g., the nucleotides themselves). As used herein, the term “mutation,” and its equivalents, may refer to a change in a gene.

The term “substitution,” and its equivalents, can refer to a nucleotide in a subject sequence that is different than an equivalent nucleotide (e.g., a nucleotide at the same position) in a reference sequence.

The term “insertion,” and its equivalents, can refer to a nucleotide in a subject sequence that is added with respect to a reference sequence.

The term “deletion,” and its equivalents, can refer to the removal of a nucleotide from a nucleotide sequence.

The terms “copy number alternation,” “CNA,” “copy number variation,” “CNV,” and their equivalents, can refer to a portion of a reference sequence that is repeated.

The terms “rearrangement of fusion,” “fusion rearrangement,” “translocation,” and their equivalents, can refer to a change in the relative position of one or more portions of a reference sequence, thereby generating a gene that was not present in the reference sequence.

The term “sequencing,” and its equivalents, may refer to a process of identifying the order and identity of monomers in a polymer chain, such as the order and identity of nucleotides in a DNA or RNA molecule. The terms “whole genome sequencing,” “WGS,” “full genome sequencing” and their equivalents, may refer to the process of sequencing an entire genome of a subject, including the introns and exons of the genes of the subject. The terms “whole exome sequencing,” “WES,” and their equivalents, may refer to the process of sequencing all exomes of a subject. The term “targeted sequencing,” and its equivalents, may refer to the process of sequencing a portion of the genome of a subject, such as sequencing a single gene of the subject. Various techniques can be utilized to sequence a DNA or RNA molecule, such as massively parallel sequencing (MPS), nanopore sequencing, direct sequencing, Sanger sequencing, or next generation sequencing (NGS). An apparatus configured to perform NGS is referred to as a “next generation sequencer.” In various cases, sequencing is performed on physical molecules (e.g., RNA or DNA) and is used to generate data.

The terms “massive parallel sequencing,” “massively parallel sequencing,” “MPS,” and their equivalents, may refer to a technique for simultaneously performing multiple reactions that can be used to identify the order and identity of monomers in multiple polymer chains. In particular cases, massive parallel sequencing can be performed using sequencing-by-synthesis on clonally amplified DNA molecules that are located in spatially separated regions, which are individually monitored by sensors.

The term “nanopore sequencing,” and its equivalents, may refer to a technique for identifying the order and identity of monomers in a polymer chain by transporting the polymer chain from a first space to a second space, wherein the first space and the second space are separated by a substrate, by directing the polymer chain through a small hole (known as a “nanopore”) embedded in the substrate, and monitoring a relative electrical signal (e.g., a voltage or current) between the first space and the second space. The electrical signal, for instance, can be detected by sensors disposed in the first space and the second space.

The terms “next generation sequencing,” “next-generation sequencing,” “NGS,” and their equivalents, may refer to any sequencing technology that was developed after Sanger sequencing. MPS and nanopore sequencing are examples of NGS.

The term “read depth” and its equivalents may refer to the number of times that a specific genomic site is sequenced during a sequencing run.

The term “locus,” and its equivalents, may refer to a specific location of one or more nucleic acid molecules on a chromosome, genome, pangenome, or the like. In some cases, a locus refers to a location of a gene, genetic marker, or other sequence is located on a chromosome. The plural form of “locus” is “loci.”

The term “endpoint,” and its equivalents, may refer to one or more bases located at a terminus of a nucleic acid molecule fragment. When a fragment is aligned with a reference genome, a “right” or “lower” endpoint of the fragment may correspond to the largest coordinate in the reference genome that is aligned with the fragment. A “left” or “upper” endpoint of the fragment may correspond to the smallest coordinate in the reference genome that is aligned with the fragment.

The term “genomic position,” and its equivalents, may refer to a molecular location of one or more base pairs within a reference genome. In some cases, the molecular location is defined by the chromosome on which the base pair(s) is located, the arm of the chromosome on which the base pair(s) is located, the distance (e.g., in base pairs) between the base pair(s) and the centromere of the chromosome, a coordinate of the base pair(s) within the genome, some other way of defining the unambiguous position of the base pair(s) within the genome, or any combination thereof.

The term “sensor,” and its equivalents, may refer to a physical device or other apparatus that is configured to detect one or more detection signals.

The term “detection signal,” and its equivalents, may refer to a physical signal that can be identified, characterized, or otherwise perceived by a sensor.

The term “sequence read data,” and its equivalents, may refer to data that is indicative of an order and identity of monomers in a polymer, such as the order and identity of nucleotides in a DNA or RNA sequence. In various implementations, sequence read data is generated via a sequencing operation.

The term “ligating,” and its equivalents, may refer to a process of joining two molecules together, for example, with a chemical bond.

The term “adapter,” and its equivalents, may refer to an oligonucleotide that can be ligated to a target nucleic acid molecule. In various cases, an adapter prepares the target nucleic acid molecule for sequencing.

The term “bait molecule,” and its equivalents, may refer to a nucleic acid molecule having a region that is complementary to a region of a target molecule (e.g., cfDNA). A bait molecule includes, for instance, a nucleic acid molecule that can hybridize to (i.e., is complementary to) a target molecule can be used to capture the target molecule. In some instances, the bait molecule is a capture oligonucleotide (or capture probe). In some instances, the bait molecule is suitable for solution phase hybridization to the target molecule. In some instances, the bait molecule is suitable for solid phase hybridization to the target molecule. In some instances, the bait molecule is suitable for both solution-phase and solid-phase hybridization to the target molecule. The design and construction of bait molecules is described in more detail in, e.g., International Patent Application Publication No. WO 2020/236941.

The term “amplifying,” and its equivalents, may refer to a process of generating copies of a target molecule, such as a nucleic acid molecule.

The term “hybridization,” and its equivalents, may refer to a process by which two complementary single-stranded nucleic acid molecules bind to one another, thereby forming a double-stranded nucleic acid molecule. In certain examples, the double-stranded nature of the nucleic acid molecule is maintained under stringent hybridization conditions. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C.

The term “complementary,” and its equivalents, may refer to a state of two single-stranded nucleic acid molecules with respective sequences that cause the nucleic acid molecules to spontaneously hybridize to one another. One nucleic acid molecule, for instance, may have a sequence that causes each nucleic acid to hydrogen bond to a respective nucleic acid in the other nucleic acid molecule.

The terms “therapy,” “treatment,” and their equivalents, may refer to a composition or process that can be used to remediate a health problem. Cancer therapies (also referred to as “anticancer therapies”), for instance, include surgery, radiotherapy (e.g., a radiation therapy), chemotherapy, immunotherapy, cell-based therapies, and the like. Examples of cancer therapies include abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), Lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximabcmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecanhziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), and combinations thereof. Examples of cancer therapies also include targeted antibody-based therapies (antibody-drug conjugates, antibody-radioisotope conjugates, and targeted immune cell therapies (e.g., immune effector cells genetically modified to express a chimeric antigen receptor (CAR).

The term “treatment-responsive,” and its equivalents, may refer to a type of cancer cells that can be substantially killed using a predetermined type of therapy. For example, cancer cells of a subject may be responsive to a particular treatment if, after the subject is administered the treatment, the cancer cells are diminished by a particular progression level (e.g., radiographic progression level, marker-based progression level, such as prostate-specific antigen (PSA) progression, etc.). Accordingly, the responsiveness of the cells to the type of therapy may indicate the effectiveness of that therapy.

The term “treatment-resistant,” and its equivalents, may refer to a type of cancer that cannot be substantially killed using a predetermined type of therapy.

The term “metastasis profile,” and its equivalents, may refer to a propensity of a type of cancer to metastasize into one or more differentiated tumor types besides the cancer's tissue origin. In some implementations, the metastasis profile can further indicate the type of tissue in which the cancer can or is likely to metastasize.

The term “survivability,” and its equivalents, may refer to an indication of whether a subject will, or is predicted to, be alive at a particular point in time. A subject's survivability, for instance, may be dependent on a type of condition experienced by the subject. In some cases, survivability is defined based on a date of diagnosis (e.g., a likelihood that a subject will be alive six months after diagnosis).

The term “clinical trial,” and its equivalents, may refer to a research study used to evaluate a hypothesis based on participation by one or more subjects. In various examples, a clinical trial can be used to assess the efficacy and/or safety of a proposed therapy. A clinical trial may be performed in furtherance of approval of a treatment by a regulatory authority (e.g., the United States Food & Drug Administration (FDA)).

The terms “cancer stage,” “stage,” and their equivalents, may refer to number indicating the spread of cancer throughout the body.

The terms “cancer grade,” “grade,” and their equivalents, may refer to a number indicating the appearance and behavior of cancer cells. Low-grade cancer cells (e.g., grade 1) appear similarly to non-cancer cells, and are predicted to grow and spread slowly. High-grade cancer cells (e.g., grade 4) appear abnormal compared to non-cancer cells, and are predicted to grow and spread relatively fast.

The terms “genomic age,” “genetic age,” and their equivalents, may refer to a subject's apparent age reflected by one or more biomarkers (e.g., epigenetic biomarkers, such as DNA methylation patterns). The “Horvath clock,” discussed in Horvath & Raj, 19 Nature Reviews Genetics 371-48 (2018), which is incorporated by reference herein in is entirety, is one example of characterizing genomic age.

The term “type,” “condition type,” and its equivalents, may refer to a collection of characteristics that are diagnosable as a distinct condition. The term “cancer type,” for instance, may refer to the cell type from which the cancer originated, the anatomical or physiological location of the cancer cells, or some other group of characteristics to clinically define an instance of cancer. The term “subtype,” for instance, refers to a more specific grouping of characteristics within a condition type.

The terms “machine learning,” “ML,” “computer learning,” “artificial intelligence,” and their equivalents, may refer to the use of a computing devices to learn patterns in training data. The process of learning these patterns may be referred to as “training.” In particular cases, one or more computing devices may perform machine learning by executing a machine learning model. As used herein, the terms “machine learning model,” “ML model,” and their equivalents, may refer to data encoding instructions that, when executed by at least one computing device, causes the at least one computing device to learn patterns in training data by optimizing one or more metrics, values, or other types of parameters. After training, an ML model, when executed by at least one computing device, causes the at least one computing device to utilize the optimized parameters in order to perform one or more tasks.

The terms “convolutional neural network,” “CNN,” and their equivalents, may refer to an ML model configured to identify features in input data by performing a series of convolutions or cross-correlations on the input data with multiple kernels (also referred to as “filters”). In various cases, the input data for a CNN is in the form of an image. In various cases, a CNN is defined according to multiple layers (also referred to as “blocks”), which may be arranged in parallel and/or series, wherein each layer is defined according to a kernel. Each layer, for instance, corresponds to a convolution and/or cross-correlation operation between the input data for the layer and the kernel that defines the layer. The output of each layer is provided as input data for a subsequent layer or is output from the CNN. In some cases, individual layers further define pooling and/or normalization functions.

The term “image,” and its equivalents, may refer to 2D or 3D array of data indicative of an array of pixels or voxels. A “digital image,” for instance, refers to digital data indicative of an image.

The terms “transform,” “data transform,” and their equivalents, may refer to a process for converting a dataset from one domain to another domain. In various cases, transforms are reversible. Data that has been generated as a result of a transform may be referred to as “transformed data.”

The term “domain,” and its equivalents, may refer to a set of possible inputs and/or a set of independent variables of a function or dataset. In some cases, if a dataset includes ordered pairs of first and second elements, wherein the second elements are respectively dependent on the first elements, then the domain of that dataset includes the first elements.

The term “peak,” and its equivalents, may refer to a local or absolute minimum within a dataset or function.

The term “trough,” and its equivalents, may refer to a local or absolute minimum within a dataset or function.

The term “distance metric,” and its equivalents, may refer to a level of similarity between a first dataset or function and a second dataset or function.

The term “artifact,” and its equivalents, may refer to an error in the perception or representation of information in a dataset.

The term “filter,” and its equivalents, may refer to a system that performs one or more mathematical operations on a signal or dataset in order to reduce or enhance aspects of the signal or dataset. In some cases, a filter can be used to remove an artifact from the dataset.

### Description of Example Implementations

Various implementations of the present disclosure will now be described with reference to the accompanying Figures.

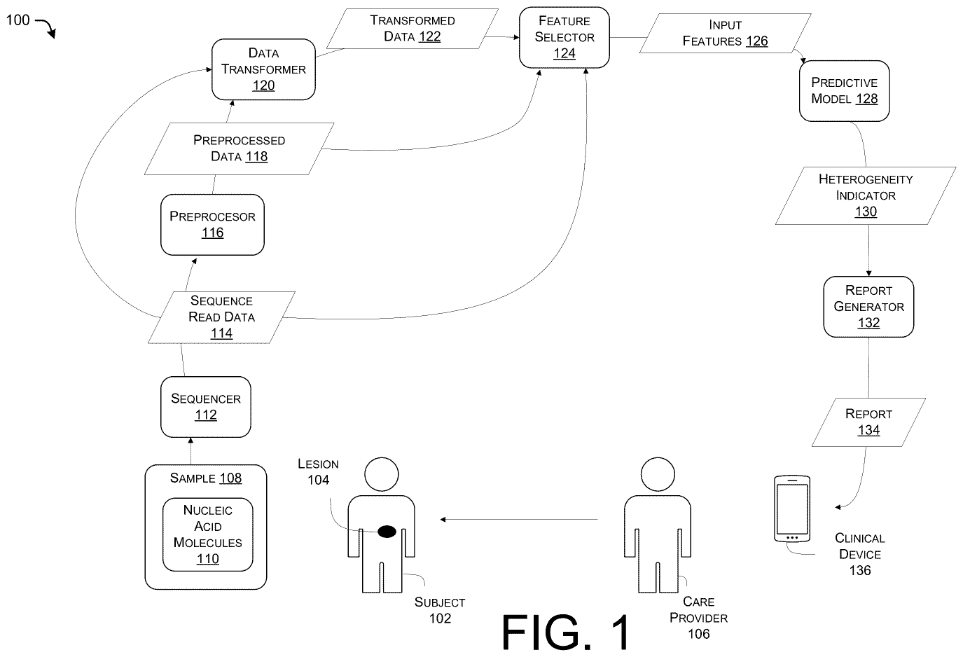

FIG. 1 illustrates an example environment 100 for predicting a tumor heterogeneity of a subject 102 based on fragmentomic features of the subject 102. In some cases, the subject 102 lacks any apparent disease or other pathological condition. For example, the subject 102 may present to a clinical environment for a medical assessment of the subject 102, such as an evaluation of the general health or well-being of the subject 102. In various cases, the subject 102 presents to the environment 100 as part of a screening assessment for cancer. For instance, the subject 102 may schedule an appointment in the environment 100 based on an age, demographic, or a family history of cancer of the subject 102, rather than in response to any symptom or suspected condition.

In various implementations, the subject 102 has a disease or a suspected disease. The subject 102, for instance, may present to the clinical environment with a lesion 104. In various cases, the lesion 104 may be a tumor that includes cancer cells. According to various examples, the subject 102 has one or more types of cancer, such as adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof.

In some embodiments, the subject 102 has a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the subject 102 has acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), a non-small cell lung cancer KRAS (+/−G12C), a non-small cell lung cancer TMB-H, a non-small cell lung cancer MET exon 14 skipping, a non-small cell lung cancer ERBB2 inframe indel, a non-small cell lung cancer EGFR exon 20 indel, a neurotrophic tyrosine receptor kinase (NTRK)-positive cancer, ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In various cases, a care provider 106 (also referred to as a “healthcare provider”) is responsible for diagnosing and/or treating the subject 102. According to some implementations, the lesion 104 may be initially identified using a noninvasive technique. For example, the lesion 104 may be visualized using an imaging modality, such as ultrasound, x-ray, computed tomography (CT), magnetic resonance imaging (MRI), positron emission tomography (PET), single photon emission CT (SPECT), or any combination thereof. However, even noninvasive techniques are inappropriate for screening examinations performed before the subject 102 has any symptoms. For instance, the cost and potential harm (e.g., radiation exposure, in the case of x-ray or CT imaging) of noninvasive techniques outweigh the limited chance of identifying the lesion 104 for a population of individuals being evaluated in a pre-disease screening context.

Moreover, even if noninvasive techniques are used to visualize the lesion 104, the care provider 106 may identify the presence of the lesion 104 but may be unable to determine whether the lesion 104 is a cancerous tumor using noninvasive diagnostic methodologies. In some cases in which the lesion 104 is a tumor, the care provider 106 may be unable to identify whether the tumor is metastatic or benign, or may be unable to otherwise categorize the tumor.

In various implementations, the care provider 106 is unable to accurately identify the tumor heterogeneity of the subject 102 based solely on noninvasive diagnostic techniques. In various cases, the care provider 106 cannot conclusively determine whether the subject 102 has a type of cancer based on noninvasive diagnostic techniques. For example, the care provider 106 is unable to identify a type of the lesion 104 (e.g., a tumor) using imaging techniques. The care provider 106 may be unable to identify a characteristic of a subject presenting with a disease (e.g., cancer), wherein the characteristic is determinative of, or at least correlated with, an effectiveness of at least one therapy at treating the disease, an ineffectiveness of at least one therapy at treating the disease, a survivability (e.g., a likelihood that the subject will survive by a predetermined date or time), an expected quality of life, at least one predetermined symptom, at least one comorbidity, another factor relevant to the prognosis associated with the disease, or any combination thereof.

The care provider 106 could identify the tumor heterogeneity of the subject 102 using histochemistry and/or immunohistochemistry. For instance, the care provider 106 could surgically remove a tissue sample from the lesion 104 and/or review the tissue sample using histochemistry and/or immunohistochemistry. However, attempting to classify the lesion 104 using these techniques has several drawbacks. First, the tissue sample may not be classifiable using conventional histological techniques, such as conventional immunohistochemical staining and review. Second, it is unlikely that the single care provider 106 would be trained to perform the tissue biopsy (which would be performed by a surgeon), to administer anesthesia to the subject 102 during the tissue biopsy (which would be performed by an anesthesiologist), and the analysis of the tissue biopsy (which would be performed by a trained pathologist), such that the classification would utilize multiple highly trained care providers. Even if the lesion 104 was classifiable by these means, the coordinated efforts of these care providers could delay classification of the lesion 104 and could cause significant expense to the subject 102. In various examples, the delay in classification could cause significant emotional hardship to the subject 102, who could be prevented from receiving an informed prognosis for weeks. The delay in classification could delay administration of a therapy to the subject 102 in order to treat the tumor, which could cause lasting harm to the subject 102, particularly in cases in which the lesion 104 is representative of an aggressive form of cancer. Further, the classification would be limited to phenotypic tumor heterogeneity, and the care provider 106 may not be able to identify genomic tumor heterogeneity using this analysis.

The care provider 106 may review the tissue sample using sequencing-based approaches. For instance, the care provider may extract and sequence genomic DNA from the tissue sample using, in various cases, a sequencing technique (e.g., Sanger sequencing, next-generation sequencing, etc.). However, the tissue sample may not be representative of the entire lesion 104, and accordingly, the heterogeneity of the tissue sample may not be representative of the heterogeneity of the lesion 104. Accordingly, the tumor heterogeneity may be incorrectly classified using these techniques.

In various implementations of the present disclosure, the tumor heterogeneity of the subject 102 can be determined without performing histochemistry and/or immunohistochemistry. For instance, a sample 108 is obtained from the subject 102. In some examples, the sample 108 includes a tissue biopsy sample. For instance, the sample 108 is obtained by removing cells from the lesion 104 and from the subject 102. In some cases, the tissue biopsy sample is surgically excised from the subject 102. In some cases, the sample includes a liquid biopsy sample. The liquid biopsy sample 108, for instance, includes blood, plasma, cerebrospinal fluid, sputum, stool, urine, lymphatic fluid, saliva, or some other fluid obtained from the body of the subject 102. In some cases, a blood sample is obtained intravenously from the subject 102. The liquid biopsy sample 108, according to various examples, is a plasma sample obtained from the blood of the subject 102. The liquid biopsy sample 108, for instance, can be obtained in a minimally invasive procedure, which could be performed by a medical technician rather than a surgeon.

The sample 108 includes nucleic acid molecules 110. According to some examples, the nucleic acid molecules 110 include genomic DNA (gDNA). For instance, the nucleic acid molecules 110 include chromosomal DNA that is located in, or extracted from, cells in the sample 108. According to some cases, the DNA is extracted from nuclei and the cells in the sample 108 using mechanical shearing and/or the introduction of a chemical (e.g., a detergent). The DNA may be subsequently isolated from proteins and other cellular materials. In some implementations, the nucleic acid molecules 110 indicate an entire genome of the subject 102 and/or the lesion 104. Thus, a genome of the subject 102 and/or the lesion 104 can be determined by sequencing the DNA in the nucleic acid molecules 110.

In some examples, the nucleic acid molecules 110 include RNA. In some implementations, the nucleic acid molecules 110 include messenger RNA (mRNA), microRNA, non-coding RNA, functional RNA, or any combination thereof. Various RNA in the nucleic acid molecules 110 may be indicative of proteins expressed in the cells of the subject 102 and/or the lesion 104.

In various implementations, the sample 108 includes cell-free DNA (cfDNA). In examples in which the subject 102 has cancer (e.g., the lesion 104 is a cancerous tumor), the cfDNA, for instance, includes circulating tumor DNA (ctDNA) and/or non-ctDNA. In cases wherein the lesion 104 is a tumor, cancer cells within the lesion 104 will lyse and release the ctDNA into the bloodstream of the subject 102. These cancer cells, for example, include circulating tumor cells (CTCs). Further, other cells additionally shed non-ctDNA into the bloodstream of the subject. In general, the cfDNA includes fragments with lengths that are in a range of 1 to 500, 3 to 500, or 100 to 500 bases long. For instance, the cfDNA includes fragments that are about 170 bases long and/or fragments that are about 340 bases long. For example, the cfDNA includes fragments that are 100 to 240 bases long and/or fragments that are 270 to 410 bases long. In some examples, the sample 108 includes hematopoietic stem cells (HSCs). In various implementations, mutations in HSCs are associated with blood cancers, and HSCs can differentiate into cancer stem cells (CSCs), which contribute to tumor heterogeneity.

In various cases, the sample 108 is transported to a location that is remote from the subject 102 for further processing. For example, the sample 108 is removed from the subject 102 in a clinical environment (e.g., a hospital) and is then transported to a remote laboratory for further testing and analysis.

A sequencer 112 is configured to generate sequence read data 114 indicating the sequences of the nucleic acid molecules 110. The sequencer 112, for instance, includes one or more devices that are configured to generate the sequence read data 114 by processing at least a portion of the sample 108. In some cases, the nucleic acid molecules 110 are extracted from the sample 108. The extraction can be performed by the sequencer 112, by another device, manually (e.g., by a laboratory technician), or any combination thereof. Any appropriate extraction method known to those of ordinary skill in the art can be utilized.

In various cases, the sequencer 112 is configured to perform one or more processes (e.g., chemical reactions) on the nucleic acid molecules 110 in order to prepare the nucleic acid molecules 110 for sequencing. For instance, the sequencer 112 may ligate adapters onto the nucleic acid molecules 110 and/or amplify the nucleic acid molecules 110, such that numerous copies of the ligated nucleic acid molecules 110 are available for sequencing. Examples of the adapters include, for example, amplification primers, flow cell adapter sequences, substrate adapter sequences, or sample index sequences. The nucleic acid molecules 110 (e.g., the ligated nucleic acid molecules 110) may be amplified by generating multiple copies of the nucleic acid molecules 110 using one or more techniques such as polymerase chain reaction (PCR), a non-PCR amplification technique, or an isothermal amplification technique. In some cases, the sequencer 112 is configured to perform whole exome sequencing (WES) on the nucleic acid molecules 110.

The sequencer 112 may identify the length, position, and identity of the bases in the nucleic acid molecules 110 by sequencing the nucleic acid molecules 110 (e.g., the amplified and/or ligated nucleic acid molecules 110). In various cases, the sequencer 112 is a next-generation sequencer configured to perform next-generation sequencing (NGS) on the nucleic acid molecules 110. In various implementations, the sequencer 112 utilizes first-generation sequencing (e.g., Sanger sequencing), second-generation sequencing (e.g., massive parallel sequencing), third-generation sequencing (e.g., nanopore sequencing), or a combination thereof. In some cases, the sequencer 112 is configured to sequence substantially all of the nucleotides of all of the nucleic acid molecules 110 fragments obtained from the sample 108. In some examples, the sequencer 112 is configured to perform targeted sequencing. For instance, the sequencer 112 may determine whether the nucleic acid molecules 110 fragments contain one or more predetermined sequences at one or more genomic locations.

In various cases, the sequencer 112 includes one or more sensors that are configured to detect physical signals (also referred to as “detection signals”) that are indicative of the nucleotide sequences of the nucleic acid molecules 110. The sequencer 112 may perform sequencing-by-synthesis. For example, the sequencer 112 may include one or more optical sensors configured to detect optical signals emitted from fluorescently tagged nucleotide triphosphates (NTPs) that are joined together in a synthesized DNA strand using the ligated nucleic acid molecules 110 as templates. The optical signals detected by the optical sensor(s), for instance, are indicative of the sequences of the nucleic acid molecules 110. The sequencer 112 may perform nanopore sequencing. In various cases, the sequencer 112 includes one or more electrical sensors configured to measure an electrical signal (e.g., an electrical current) across a substrate as the ligated nucleic acid molecules 110 are directed through a nanopore extending through the substrate. The electrical signal over time, in various cases, is indicative of the sequences of the nucleic acid molecules 110 in the sample 108. The sequencer 112, in various implementations, is configured to generate the sequence read data 114 as digital data based on the analog signals detected by the sensor(s). For instance, the sequencer 112 includes one or more analog to digital converters (ADCs). In various cases, the sequencer 112 includes at least one processor configured to generate the sequence read data 114.

In some implementations, the sequencer 112 performs RNA sequencing (RNA-seq) on the nucleic acid molecules 110. For example, the nucleic acid molecules 110 include RNA that is extracted from the sample 108. In some examples, the RNA in the nucleic acid molecules 110 is fragmented. In various implementations, complementary DNA (cDNA) is generated using reverse transcriptase, such that the cDNA includes sequences that are complementary to the RNA in the nucleic acid molecules 110 from the sample 108. The cDNA, according to various cases, can be sequenced using the DNA sequencing techniques described above. Accordingly, in some cases, the sequence read data 114 indicates sequences of RNA present in the sample 108, which may be indicative of the transcriptome of the subject 102 and/or the lesion 104.

In various cases, the sequencer 112 performs sequencing on a subset of the nucleic acid molecules 110. For instance, the sequencer 112 may perform targeted sequencing on portions of the nucleic acid molecules 110 that correspond to one or more predetermined genes, such as any of the specific genes described herein. Other portions of the genome may be specifically sequenced, such as promoters, hotspots, CpG sites, or other portions of the genome that are not specifically genes but have an impact on genomic expression. The sequencer 112, in some cases, may refrain from sequencing at least a portion of the nucleic acid molecules 110 that do not correspond to the subset.

The sequence read data 114, according to various instances, is in a spatial domain. For example, the sequence read data 114 may be indicative of the genomic locations of the nucleic acid molecules 110 in the sample 108. In various cases, the sequence read data 114 may be difficult to analyze directly. Although it may be possible to identify, in the sequence read data 114, attributes or other characteristics that are predictive of the condition of the subject 102, such analyses may utilize numerous computing resources.

According to some implementations, the sequence read data 114 is preprocessed by a preprocessor 116. For example, the preprocessor 116 performs one or more preprocessing steps on the sequence read data 114 to generate preprocessed data 118. In some cases, the preprocessor 116 performs normalization on the sequence read data 114. In various implementations, the preprocessor 116 performs smoothing on the sequence read data 114. For example, the preprocessor 116 is configured to assign, to a specific genomic position, an average (e.g., mean) endpoint count among endpoint counts in window surrounding the genomic position in the sequence read data 114. For example, a given genomic position in the preprocessed data 118 is assigned an average endpoint count among endpoint counts within a window of ±5, ±10, ±15, ±20, ±50, or ±100 genomic positions that are directly adjacent to the given genomic position.

In some cases, the preprocessor 116 selects a portion of the sequence read data 114 based on its relative abnormality compared to sequence read data of a population. In various cases, the population omits heterogeneous tumors or a particular tumor heterogeneity. Thus, the preprocessor 116 may select the portion of the sequence read data 114 that is most likely to be indicative of the genomic features of the subject 102 that uniquely characterize the subject 102 relative to the population. In some cases, the selected portion of the sequence read data 114 is particularly pertinent to whether or not the subject 102 has a heterogeneous tumor and/or a particular tumor heterogeneity. According to some cases, the preprocessed data 118 includes the selected portion of the sequence read data 114. In some examples, the preprocessed data omits at least some of the nonselected portion of the sequence read data 114.

In various implementations of the present disclosure, the sequence read data 114 and/or the preprocessed data 118 is output to a data transformer 120 rather than analyzed directly. The data transformer 120 is configured to generate transformed data 122 by transforming the sequence read data 114 from a first domain (e.g., the spatial domain) to a second domain that is different than the first domain. That is, the second domain is an “alternate” domain to the first domain. In some cases, the transformed data 122 includes data representing the sequence read data 114 in the second domain. In some examples, the transformed data 122 includes one or more images representing the sequence read data 114 in the second domain.

Various types of transformations can be performed by the data transformer 120. In some examples, the data transformer 120 is configured to generate the transformed data 122 by performing a Fourier transform on the sequence read data 114 and/or the preprocessed data 118. The transformed data 122, for instance, is in a frequency domain. According to some examples, the data transformer 120 is configured to perform a Fast Fourier Transform (FFT) on the sequence read data 114. In some cases, the data transformer 120 is configured to perform a continuous Fourier transform on a function representative of the sequence read data 114 and/or the preprocessed data 118. In various examples, the data transformer 120 is configured to perform a discrete Fourier transform (DFT) on the sequence read data 114 and/or the preprocessed data 118. According to some cases, the data transformer 120 is configured to perform a short-time Fourier transform (STFT) on the sequence read data 114 and/or the preprocessed data 118.

In some examples, the data transformer 120 is configured to generate the transformed data 122 using one or more other types of transforms. For example, the data transformer 120 may generate the transformed data 122 by performing a Hartley transform, a Laplace transform, a Mellin transform, a wavelet transform (e.g., a continuous wavelet transform (CWT), a discrete wavelet transform (DWT), a fast wavelet transform (FWT), a complex wavelet transform, a Newland transform, a stationary wavelet transform (SWT), a second generation wavelet transform (SGWT), a dual-tree complex wavelet transform (DTCWT), etc.), or any combination thereof, on the sequence read data 114 and/or the preprocessed data 118. In some cases, the data transformer 120 generates the transformed data 122 by generating a Taylor series or Taylor expansion of the sequence read data 114. Example transforms are described, for instance, in Farge, 24 Annu. Rev. Fluid Mech. 395-457 (1992), which is incorporated by reference herein its entirety.

According to various cases, the transformed data 122 represents at least one locus of interest indicated by the sequence read data 114. For instance, the transformed data 122 may include a second-domain mapping of a portion of the sequence read data 114 and/or the preprocessed data 118 that reflects at least one gene-of-interest of the subject 102 and/or the lesion 104, as reflected in the sequence read data 114. Examples of genes with potential relevance to a determination of whether the subject 102 has a type or subtype of cancer include ABL1, ACVR1B, AKT1, AKT2, AKT3, ALDH2, ALK, ALOX12B, AMER1, APC, APOE, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BNIP3, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CK8, CK18, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), ENO1, EP300, EPHA3, EPHB1, EPHB4, ERBB2, ERBB3, ERBB4, ERCC4, ERG, ERRFI1, ESR1, ETV1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXA1, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KLK3, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MRPL44, MSH2, MSH3, MSH6, MST1R, MT-CO1, MT-CO3, MT-ND1, MTAP, MTOR, MUTYH, MYB, MYC, MYCL, MYCN, MYD88, NBN, NDUFV2, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PGK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1,RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC16A3, SLC34A2, SMAD2, SMAD4, SMARCA4, SMARCB1, SMO, SNAIL1, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TWIST1, TYRO3, U2AF1, VEGFA, VHL, WHSC1, WHSC1L1, WT1, XPO1, XRCC2, ZNF217, or ZNF703. In some cases, the genes include at least one estrogen receptor (ER) gene and/or at least one progesterone receptor (PR) gene. In some cases, the genes include one or more of ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB. In some examples, the genes include one or more of TP53, CTNNNB1, L1CAM, PTEN, POLE, MKI67, FAT3, TAF1, ZFHX3, RPL22, SPTA1, FAM135B, CSMD3, GIGYF2, CSDE1, MLL4, ATR, CTNNB1, USH2A, LIMCH1, RRN3P2, FBXW7, CDH19, USP9X, COL11A1, BCOR, ARID1A, ZNF770, ARID5B, SLC9A11, KRAS, PNN, INPP4A, CTCF, CHD4, AMY2B, RBMX, PPP2R1A, TNFAIP6, PIK3R1, SGK1, HOXA7, METTL14, HPD, MIR1277, CCND1, MECOM, NFE2L2, or ESR1.

In some cases, characteristics of the sequence read data 114 can be more efficiently identified by preprocessing the sequence read data 114 and transforming the preprocessed data 118 into the alternate domain. Accordingly, transforming the sequence read data 114 and/or preprocessed data 118, in some examples, can greatly reduce the amount of processing resources utilized to identify the condition of the subject 102. Further, in some cases, transforming the sequence read data 114 and/or preprocessed data 118 enables new characteristics to be identified using the sequence read data 114. In some cases, the accuracy of a classification (e.g., of whether or not the subject 102 has the tumor heterogeneity) performed on the transformed data 122 is greater than if a classification is performed on the sequence read data 114 in the spatial domain, alone.

A feature selector 124 identifies input features 126 of the nucleic acid molecules 110 by analyzing the sequence read data 114, the preprocessed data 118, the transformed data 122, or any combination thereof. In various implementations, the feature selector 124 identifies, calculates, or otherwise determines the input features 126 based on the sequences of the nucleic acid molecules 110 indicated in the sequence read data 114, the preprocessed data 118, the transformed data 122, or any combination thereof. One or more types of features are identified by the feature selector 124. In various implementations, the input features 126 are genomic features. That is, the input features 126 may be derived from the sequence read data 114 in addition to the transformed data 122.

In various cases, the input features 126 are derived based on fragments in the nucleic acid molecules 110, and are therefore referred to as “fragmentomic features.” Examples of fragmentomic features include endpoint positions of the fragments in a reference genome (e.g., right endpoints, left endpoints, etc.), endpoint counts at positions within the reference genome (e.g., right endpoint counts, left endpoint counts, etc.), fragment lengths, end motifs, relative read depths of the fragments, the presence of one or more variants in the fragments, or any combination thereof. Fragmentomic features can be expressed in the spatial domain, in an alternate domain, in a preprocessed form, or any combination thereof.

In some examples, the input features 126 include at least one distance metric. For example, the feature selector 124 may generate the distance metric by comparing the transformed data 122 to pre-classified data that is in the same domain as the transformed data 122. In some cases, the pre-classified data is generated based on nucleic acid molecules obtained from one or more individuals with known presentations of the tumor heterogeneity. For example, the pre-classified data may include transformed data of an individual with the tumor heterogeneity. According to some cases, the pre-classified data is generated based on nucleic acid molecules obtained from one or more individuals with the absence of a particular condition, such as an individual without the tumor heterogeneity, an individual without a heterogeneous tumor, or an individual without cancer. In various cases, the distance metric(s) may represent a similarity between the transformed data 122 and the pre-classified data. For example, the distance metric(s) may be generated by cross-correlating and/or convolving the transformed data 122 and the pre-classified data. In some cases, the distance metric(s) include the value of a peak and/or mean of the cross-correlated and/or convolved data. According to various implementations, a magnitude of the distance metric(s) is indicative of a likelihood that the nucleic acid molecules 110 of the subject 102 reflect the tumor heterogeneity of the pre-classified data. Thus, the tumor heterogeneity of the subject 102 can be identified using the distance metric(s).

According to some implementations, the feature selector 124 performs image processing techniques in order to generate the input features 126. In some cases, the feature selector 124 generates a digital image based on the sequence read data 114, the preprocessed data 118, the transformed data 122, or any combination thereof. For example, the feature selector 124 may generate a spectrogram or other graphical representation of the transformed data 122. In some cases, the feature selector 124 generates the input features 126 by analyzing the image of the transformed data 122.

In some cases, the feature selector 124 includes a machine learning (ML) model configured to identify features of the image that are predictive of the condition of the subject 102. For instance, the feature selector 124 may include a convolutional neural network (CNN) that generates the input features 126 in response to receiving the image representative of the transformed data 122. In some examples, the pixel intensities in the image are indicative of the sequence read data 114, the preprocessed data 118, or the transformed data 122. For instance, the pixel intensities may be indicative of a distribution of the DNA fragments indicated by the preprocessed data 118. According to various examples, the CNN may include multiple blocks and/or layers that are each defined by a kernel (e.g., a digital image filter). Each block and/or layer may be configured to convolve and/or cross-correlate the kernel with pixels of an input image, thereby generating an output image. In some cases, the blocks and/or layers are arranged in series, such that the input image of one block and/or layer may be the output image of another block and/or layer. Each block and/or layer may further be defined according to a receptive field of its kernel and/or a stride size of the kernel.

In some examples, the CNN of the feature selector 124 is pretrained. For example, the values of the kernel of each block and/or layer may be optimized based on training data prior to receiving the image of the transformed data 122. In some examples, the training data includes other images of other transformed data, as well as manually obtained indications of the types of input features that the CNN is being trained to identify. The CNN, for instance, may be trained using a supervised learning technique. Because the CNN is pretrained, the CNN may be configured to output the input features 126 in response to receiving the image of the transformed data 122.

According to some examples, the feature selector 124 is configured to filter the transformed data 122. For instance, the feature selector 124 may be configured to apply one or more filters in the domain of the transformed data 122. For example, the feature selector 124 may apply a filter by convolving, cross-correlating, or multiplying the second-domain representation of the filter with the transformed data 122. By filtering the transformed data 122, in some cases, the feature selector 124 can reduce or eliminate artifact in the transformed data 122 and/or enhance one or more characteristics indicative of the input features 126 in the transformed data 122. In some cases, it may be more computationally efficient to apply the filter to the transformed data 122 in the second domain than to the sequence read data 114 or to the preprocessed data 118 in the first domain. Examples of filters include a Butterworth filter, a Chebyshev filter, a finite impulse response (FIR) filter, or an infinite impulse response (IIR) filter. In some cases, the filter applied by the feature selector 124 is a low-pass filter, a high-pass filter, or a bandpass filter. For instance, the filter may be defined by one or more cutoff frequencies.

One or more types of characteristics may be included in the input features 126. In some cases, the input features 126 are derived exclusively by the feature selector 124 based on the transformed data 122. For example, the input features 126 may include a digital image of at least a portion of the transformed data 122 and/or features derived based on the digital image. In some cases, the input features 126 include at least one peak of the transformed data 122, at least one trough of the transformed data 122, a distance metric associated with the transformed data 122, an indication of whether at least a portion of the transformed data 122 exceeds a threshold, or any combination thereof. In particular examples, the input features 126 are derived by the feature selector 124 based on a combination of the transformed data 122, the preprocessed data 118, and the sequence read data 114.

In some cases, the input features 126 include a mismatch repair deficiency (MMRD) probability score. In various cases, the MMRD probability score indicates a likelihood that one or more MMR pathways of cells in the sample 108 are ineffective at performing mismatch repair. In some implementations, the MMRD probability score is determined by determining genomic features by analyzing the sequence read data 114, inputting the genomic features into at least one trained machine learning model trained to generate the MMRD probability score based on previously analyzed data from a population omitting the subject 102. The genomic features relevant to the MMRD probability score include, for instance, a fraction unstable score, a composite Catalogue Of Somatic Mutations In Cancer (COSMIC) single-base substitution signature, a COSMIC indel signature, a copy number signature, a tumor mutational burden score, a blood-based tumor mutational burden score, a germline status for a mutation in one or more genes associated with DNA mismatch repair (MMR) (also referred to as “MMR genes”), a methylation status for the one or more MMR genes, a methylation status for one or more promoters associated with the one or more MMR genes, a methylation status of one or more enhancers associated with the one or more MMR genes, or any combination thereof. Examples of the MMR genes include, for instance, MSH2, MSH6, PMS2, or MLH1.

The input features 126, in some examples, include a copy number state of one or more genetic loci indicated by the sequence read data 114. In various implementations, a number of copies of a predetermined sequence at a given locus in the genome of the subject 102 and/or the lesion 104 (also referred to as a “copy number” of the locus) is determined. The copy number state, in various implementations, may indicate copy numbers of one or more loci in the genome of the subject 102 and/or the lesion 104. For instance, the copy number state may indicate the presence and/or amount of copies of various sequences present in the genome of the subject 102 and/or the lesion 104, which may be due to copy number variation.

According to various examples, the sequence read data 114 may represent a genome of the subject 102 and/or the lesion 104. Various portions of the sequence read data 114 are aligned with at least one reference sequence (e.g., a reference genome). The aligned data is segmented using at least one segmentation technique (e.g., a circular binary segmentation (CBS) method, a maximum likelihood method, a hidden Markov chain method, a walking Markov method, a Bayesian methods, a long-range correlation method, a change point method, or any combination thereof), thereby generating non-overlapping segments of the sequence read data 114, wherein a sequence associated with a given segment is associated with the same copy number (e.g., a number of instances in which the sequence appears in the segment). Various genetic loci are binned, or otherwise sorted, with respect to the segments of the genome of the subject 102 and/or the lesion 104. The copy number state, for instance, is representative of the respective copy numbers associated with the genetic loci. In some cases, the copy number state is dependent on (e.g., assigned based on) a major allele coverage ratio and a minor allele coverage ratio, as well as one or more copy number grid models.

In some implementations, the input features 126 include the presence or absence of a variant (e.g., a pathogenic variant) in one or more genes associated with classifying the lesion 104. In various cases, the genes include one or more of the genes with potential relevance to a determination of whether the subject 102 has a type or subtype of cancer, as listed above.

In some cases, the input features 126 are indicative of microsatellite instability (MSI). Microsatellites are highly polymorphic DNA-repeat regions. In certain examples, “microsatellite” refers to a repetitive nucleic acid having repeat units of less than about 10 base pairs or nucleotides in length. In certain examples, a microsatellite refers to a tract of tandemly repeated (i.e., adjacent) DNA motifs ranging from one to six or up to ten nucleotides, with each motif repeated 5 to 50 repeated times. During DNA replication, mutations (e.g., insertions or deletions) are more likely to be introduced at microsatellites than various other portions of the genome. In various cases, these mutations are corrected via MMR pathways. However, if the MMR pathways are impaired (e.g., the MMR genes of the hosting cell include variants that impede function), then the mutations at the microsatellites may be substantially retained. “Microsatellite instability” refers to genetic instability in the microsatellite regions. Cancer patients with microsatellite instability classified as being high (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy,” Appl Immunohistochem Mol Morphol, 26(2):e15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma. According to various examples, “MSI score” refers to an amount of instability in one or more microsatellites. For example, an MSI score can be represented as a fraction (i.e., an “MSI fraction”) of instability in the one or more microsatellites. Other types of portions of DNA may be associated with a high likelihood of mutations. In some cases, the input features 126 include a fraction unstable score, indicative of mutations in the microsatellites and other portions of the genome that are prone to mutations.

In various cases, an MSI score can be determined based on a predetermined set of repetitive loci (e.g., 2000 repetitive loci, each with a minimum of 5 repeat units of mono-, di-, and trinucleotides). By evaluating the sequence read data 114, the feature selector 124 may determine lengths of repetitive sequences corresponding to the loci. If an example locus among the loci corresponds to a predetermined repeat length, the locus is considered to be “unstable.” The MSI score, for instance, is determined by determining an amount of the unstable loci (e.g., a fraction of the unstable loci with respect to the total number of repetitive loci evaluated). In some cases, the MSI score is used to determine whether the subject 102 and/or lesion 104 is MSI-High (MSI-H). For example, MSI-H status may be applicable if the MSI score is greater than a threshold (e.g., 0.5%). Techniques for determining MSI scores are described, for instance, in Woodhouse et al., “Clinical and analytical validation of FoundationOne LiquidCDx, a novel 324-Gene cfDNA-based comprehensive genomic profiling assay for cancers of solid tumor origin,” PLoS ONE 15(9) (2020).

In some cases, the input features 126 may include an endpoint density. The left and right endpoints of naturally cleaved DNA provide information about the underlying biology of chromatin accessibility, transcription factor/protein binding, and gene expression, with the ability to distinguish cell type, tumor type, cell dependencies, and other cellular phenotypes. Endpoint density can be normalized to the bait coverage, smoothed, z-score normalized, or a combination thereof. Informative regions can be identified by comparing endpoint density between samples with a known phenotype, A or B. In some examples, informative regions (e.g., regions associated with the tumor heterogeneity condition of the subject 102) can be identified using a clustering approach (e.g., an unsupervised approach). In various cases, the endpoint density (e.g., a distribution of the endpoint density) is associated with the tumor heterogeneity of the subject 102. Tumors with heterogeneity may have evidence of multiple cell types present. In some examples, a sample associated with high tumor heterogeneity (e.g., a sample associated with more than one cell type) may have an endpoint density indicative of multiple cell types.

In some cases, the input feature 126 may include lengths of DNA fragments. In various examples, the lengths of DNA fragments correspond to the local read lengths of the DNA fragments. Tumor DNA is naturally cleaved in a different way than non-tumor DNA and these patterns of fragmentation may cause global shifts in DNA fragment length (e.g., shorter DNA fragment length). There may also be local changes in DNA fragment length. For example, in genes actively transcribed in a tumor there is more shearing of the DNA since it is highly accessible during transcription. Thus, tumor cells that have certain transcriptional pathways activated will have a particular DNA fragment length signature (e.g., pattern) in particular genomic regions. Effects are not limited to transcription but can be influenced by one or more of: nucleosome state, chromatin architecture, or transcription factor binding, which are all characteristic of cellular identity and cell state. These fragment lengths can be calculated across the regions baited during sequencing; by comparing fragment lengths in different cell states, characteristic regions associated with the tumor heterogeneity condition of the subject 102 can be identified. Tumors with more than one cell population may be considered heterogeneous.

In some cases, the input features 126 may include a combined metric based on both fragment length and endpoint information. The combination of these features may be non-linear and may provide even more information. For instance, an endpoint density by length matrix can be used to find particular signatures of at least one cell state of tumor cells of the subject 102 (e.g., a cell type).

In some cases, the input features 126 may include read depth depletion of the DNA fragments (e.g., in genomic regions spanning transcription factor binding sites). The density of reads (e.g., a number of sequenceable fragments at a genomic location) in a center of a genomic region versus the flank of the genomic region, can quantify things like transcription factor binding or promoter activity that may be associated with one or more cell states. Comparing the read depth depletion to cell state patterns (e.g., during training) enables the derivation of cell state from the read depth depletion of the DNA fragments.

In some cases, the input features 126 may include gene body depletion. Actively transcribed genes have fewer reads in the gene body compared to flanking regions. The amount of depletion can indicate level of transcription and help infer one or more cell states of tumor cells of the subject 102. For instance, genes with greater or less depletion than expected based on tumor fraction can indicate regions of higher or lower copy number state.

In some implementations, the input features 126 include a mutation signature. In various cases, a mutational signature can represent an amount and/or identity of mutations (e.g., insertions, deletions, double-base substitutions, single-base substitutions, or any combination thereof) indicated in the nucleic acid molecules 110 from the subject 102. In some cases, the mutational signature indicates an amount (e.g., number or percentage) of individual classes of base substitutions present in the nucleic acid molecules 110. For instance, the classes include single-base substitutions including C>A, C>G, C>T, T>A, T>C, and T>G. A mutational signature can be derived by comparing the sequences indicated in the sequence read data 114 to at least one reference sequence, such as a reference genome. For example, the input features 126 may include a COSMIC mutational signature, such as a COSMIC indel signature. In some cases, the input features 126 include a single-base substitution signature.

In various examples, the input features 126 include a tumor mutational burden (TMB) score. Tumor mutational burden (TMB) is a measure of the number of mutations carried by tumor cells. By comparing DNA sequences from a patient's healthy tissues and tumor cells, the number of acquired somatic mutations present in tumors, but not in normal tissues, may be determined. In some instances, driver mutations may be excluded from a TMB calculation. In certain examples, “tumor mutational burden” or “TMB score” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self. In addition, germline variants do not reflect the biology of somatic mutation for the purposes of TMB determinations. In various cases, driver mutations are excluded from a TMB calculation.

In some cases, the input features 126 include the presence, amount, type, or any combination thereof, of one or more hotspot mutations. Hotspots, for instance, can refer to loci in the genome of the subject 102 and/or the lesion 104 that are prone to mutation. Examples of hotspots include CpG islands, microsatellites, centromeric DNA, telomers, subtelomeric regions, common fragile sites, palindromic AT-rich repeats (PATRRs), G-quadruplexes, R-loops, and the like.

Hotspot mutations give rise to oncological outcomes. PhyloP, SIFT, Grantham, COSMIC and PolyPhen-2 are in silico tools that can be used to assess pathogenicity of identified variants. Exemplary hotspot genes and mutations include EGFR exon 19 activating mutation, EGFR exon 19 deletion, EGFR exon 19 insertion, EGFR exon 19 sensitizing mutation, EGFR exon 20 activation mutation, EGFR exon 20 insertion, EGFR G719 mutation, EGFR L858R mutation, EGFR L861 mutation, EGFR S768 mutation, EGFR T790M mutation, C797 mutation, KIT activating mutation, KRAS activating mutation, MET activating mutation, NRAS activating mutation, PMS2 promoter mutations, among many others. Hotspot mutations also occur in the following genes: AKT2, BRCA1, BRCA2, ERC1, NSD1, POLH, PPM1G, PTEN, RAD18, RAD51, RAD51B, RB1, TERT, TP53, TP53Bp1, ALK, ARMT1, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1, TACC3, TERF2, TMEM106B, UBE2L3, USP10, WRDR48, YAP1, ZEB2, and ZMYND8.

The input features 126, in particular examples, include the presence, amount, type, or any combination thereof, of one or more aneuploidy events. For instance, the input features 126 may indicate whether the subject 102 and/or the lesion 104 includes one or more extra chromosomes (e.g., greater than a pair of 23 chromosomes), one or more missing chromosomes (e.g., less than the pair of 23 chromosomes), one or more extra chromosome arms, or one or more missing chromosome arms.

In some implementations, the input features 126 include a tumor purity of the sample 108. In various implementations, the tumor purity represents an amount of the nucleic acid molecules 110 that originate from a tumor (e.g., the lesion 104) with respect to a total amount of the nucleic acid molecules 110 in the sample 108. Tumor purity can be estimated, for instance, based on a presence or amount of somatic copy-number alterations (SCNA), single-nucleotide variants (SNVs), minor allele frequency (MAF), or any combination thereof, observed with respect to the sequence read data 114.

In some cases, the input features 126 include additional biomarker data. That is, the input features 126 may include non-genomic features. For instance, input features 126 may include data indicating at least one of a histological and/or immunohistological image of the sample 108 or another sample of the lesion 104, a genomic alteration, or a viral status of the subject 102 and/or lesion 104. The additional biomarker data may be generated based on the sample 108, medical images, or other samples obtained from the subject 102. In some cases, the additional biomarker data includes an image of a stained section of the lesion 104. For instance, the stained section is stained with hematoxylin and eosin (H&E) and/or at least one immunostain.

To categorize the tumor heterogeneity of the subject 102, a predictive model 128 is configured to generate a heterogeneity indicator 130 based on the input features 126. The predictive model 128, for example, may include one or more mathematical and/or computer-based models that are configured to predict the heterogeneity indicator 130 based on the input features 126. For instance, the predictive model 128 may include a regression model, threshold rule, confidence interval, or other type of statistical model capable of categorizing the cancer based on the input features 126. In various cases, the predictive model 128 includes at least one classifier configured to generate the heterogeneity indicator 130 based on the input features 126.

In various implementations, the predictive model 128 includes at least one trained ML model configured to output the heterogeneity indicator 130 in response to receiving the input features 126 in input data. For example, parameters of the ML model(s) may have been previously optimized based on training data including features of individuals within a population omitting the subject 102. For instance, the ML model(s) was trained using an unsupervised or semi-supervised learning technique, wherein the parameters were optimized to categorize (e.g., cluster) the features of the population. In some cases, the ML model(s) was trained using a supervised learning technique, wherein the training data further included ground truth tumor heterogeneity conditions of the individuals in the population, such that the parameters were optimized to minimize a loss between predicted tumor heterogeneity conditions generated by the ML model(s) based on the features of the population and the ground truth tumor heterogeneity conditions of the cancers experienced by the individuals in the population. To increase training robustness, the population represented by the training data may include individuals without the tumor heterogeneity, individuals without a heterogeneous tumor, individuals without cancer, as well as individuals with a variety of types of presentations of the tumor heterogeneity. Various types of ML models can be included in the predictive model 128, such as a neural network (e.g., a CNN, which may be different than a CNN in the feature selector 124), a nearest-neighbor model, a regression analysis model, a clustering model, a principal component analysis model, a gradient boosting model, a random forest, or any combination thereof. In some cases, the predictive model 128 includes a hybrid model, that includes multiple types of ML models. For instance, the predictive model may include a CNN and a clustering model.

In particular examples, the predictive model 128 includes a clustering model. In various implementations, the clustering model is pre-trained based on training data that includes population features. According to various implementations, the population features include genomic features and/or additional biomarker data of the population. In some cases, the population features further include one or more known tumor heterogeneity conditions and/or prognostic classifications of the population. In various implementations, at least one computing device is configured to cluster the population features. The clustering model, for instance, stores, includes, or otherwise indicates the determined clusters.

In various examples, the population characteristics are defined in a multi-dimensional feature space. In various cases, the feature space has n dimensions (e.g., a dimensionality value of n), wherein n corresponds to the number of feature types included in the population feature. For example, one dimension may correspond to a number of peaks in the transformed data 122 that exceed a threshold, another dimension may refer to a distance metric representing a similarity between the transformed data 122 and pre-classified transformed data based on a sample obtained from an individual with a particular type of cancer, and so on. In various cases, data objects representing the population features of the population are plotted or otherwise defined in the feature space. In some examples in which n is greater than two, the data objects are projected onto an m-dimensional feature space using multi-dimensional scaling, wherein m is between 1 and n−1 (inclusive). Multi-dimensional scaling can be achieved using various techniques. For instance, multi-dimensional scaling can be performed using at least one of a statistical method (e.g., t-distributed stochastic neighbor embedding (t-SNE), uniform manifold approximation and projection (UMAP), representation learning (e.g., principal component analysis (PCA), independent component analysis (ICA), etc.), ML-based latent space learning (e.g., autoencoders, transformers, generative adversarial networks, etc.). Accordingly, in some cases, the data objects can be visualized in a Cartesian coordinate system.

Within the feature space (whether it has two or more than two dimensions), the data objects are separated from each other by distances. Various types of distances can be utilized in implementations of the present disclosure. For example, the distances may include Euclidian distances, Manhattan distances, Hamming distances, Minkowski distances, Chebyshev distances, or any combination thereof.

Various clustering techniques can be utilized to generate the clustering model. For instance, the clusters may be generated using k-means clustering, density-based clustering, centroid-based clustering, spectral clustering, distribution-based clustering, hierarchical clustering, or any combination thereof. In some implementations, the clustering model is generated by performing hierarchal clustering on the data objects representing the population features. In various cases, the clusters include two or more data objects that are within proximity of each other (e.g., within a predetermined distance of one another) in the feature space. For instance, a cluster may include two or more data objects that are within a predetermined distance (e.g., Euclidian distance) of one another in the feature space. In some implementations, a data object is included in a cluster if the data object is within an appropriate distance of a linkage criterion representing one or more data objects that are already defined within the cluster. Various implementations of the present disclosure utilize one or more linkage criteria, such as a single-linkage criterion, a complete-linkage criterion, an average-linkage criterion (e.g., a weighted average criterion, an unweighted average criterion), a centroid-linkage criterion, a median linkage criterion, a Ward linkage criterion, a minimum error sum of squares criterion, a min-max criterion, a Hausdorff linkage criterion, a medoid linkage criterion, a minimum energy clustering criterion, or any combination thereof.

In some cases, agglomerative clustering is used to generate the clusters. For example, initially, each data object is defined within the feature space without clustering. Subsequently, pairs of adjacent data objects may be clustered together. In some examples, the process of generating a cluster based on independent data objects in a feature space, or of adding a data object to an existing cluster, may be referred to as “merging.”

In some examples, divisive clustering is used to generate the clusters. For example, the data objects may be defined into a single cluster in the feature space. Subsequently, the single cluster may be divided into multiple clusters. In some instances, the process of dividing a preliminary cluster into multiple subsequent clusters, or of removing a data object from a cluster, may be referred to as “splitting.”

In various cases, each cluster is defined according to a boundary (also referred to as a “border”). In some implementations, data objects outside of the boundary of a cluster are not part of the cluster. Data objects inside of the boundary of the cluster are part of the cluster. Depending on the data objects, the linkage criterion, the feature space, and other characteristics of the training data, the clusters may have irregular shapes within the feature space. In various cases, the clustering model includes the boundaries of the clusters generated based on the data objects defined by the population features.

According to various cases, each cluster in the clustering model is associated with one or more characteristics. The characteristic(s), for instance, are associated with the presence or absence of the tumor heterogeneity in the samples associated with the cluster. In some cases, at least one characteristic is defined in at least one dimension of the feature space, such that the clusters are defined according to the tumor heterogeneity condition(s). In some examples, the population features used to define the clusters include characteristics that are beyond the mere categorization of the presence or absence of the tumor heterogeneity in the population. Once the clusters are generated based on non-tumor heterogeneity features (e.g., genomic features, such as fragmentomic features, and/or additional biomarker data), characteristics associated with the clusters are subsequently determined. For example, an example cluster may be defined based on the data objects representing the non-condition population features of m members of the population, wherein m is an integer that is greater than one. In various cases, characteristics of the m members of the population are determined. Common characteristics of the population (e.g., the presence or absence of the tumor heterogeneity) are determined. For example, if greater than a threshold number of the m members have the tumor heterogeneity that is resistant to a predetermined therapy, than resistance to the predetermined therapy may be associated with the example cluster. In various cases, each cluster may be labeled with, or otherwise associated with, one or more characteristics, such as one or more pathological and/or nonpathological conditions. The one or more tumor heterogeneity conditions and/or prognostic features associated with a given cluster form the condition associated with the cluster. In various cases, each cluster in the clustering model is associated with a particular tumor heterogeneity state.

In various implementations, the tumor heterogeneity the subject 102 is categorized by comparing the input features 126 of the subject 102 to the clusters in the clustering model. The heterogeneity indicator 130 is determined based on a comparison between the input features 126 and the clusters in the clustering model. In various cases, a data object defined by the input features 126 of the subject 102 is defined in the feature space of the clustering model. The clustering model, for instance, may determine that the data object is present within the boundary of a particular cluster that was previously defined based on the training data. In some cases, the clustering model determines that the data object is associated with a particular cluster based on a distance between the data object and the particular cluster in the feature space. In some cases, the distance is at least one of a Euclidian distance, a Manhattan distance, a Hamming distance, a Minkowski distance, a Chebyshev distance, or any combination thereof. For instance, the clustering model determines that the distance between the data object and the boundary and/or a centroid of the particular cluster is below a threshold distance. In some examples, the clustering model classifies the condition of the subject 102 into a classification associated with the particular cluster by determining that a distance between at least one data object corresponding to the population features in the cluster is below a threshold distance.

In various cases, the heterogeneity indicator 130 of the sample 108 is generated using the input features 126 and the clustering model. For example, the clustering model may determine that the subject 102 is associated with one or more tumor heterogeneity conditions and/or prognostic features associated with the cluster in which the input features 126 belong. In various examples, the prognostic features may include the predicted presence or absence of tumor heterogeneity, at least one predicted cell type of the lesion 104 of the subject 102, a predicted tumor evolution of the subject 102, a predicted tumor progression of the subject 102, a predicted metastasis profile of the subject 102, or a predicted survivability of the subject 102. The predicted tumor evolution, in some examples, may include predicted clones associated with the lesion 104 and/or indications of characteristics associated with the predicted clones. For instance, the predicted tumor evolution may indicate that a predicted clone is resistant to a treatment (e.g., a chemotherapy). The predicted tumor progression may indicate a rate of growth of the lesion 104. The predicted metastasis profile may indicate a time (e.g., a date, a time range) when the lesion 104 will metastasize. In some examples, the heterogeneity indicator 130 include a likelihood that the lesion 104 will metastasize (e.g., to the lymph nodes, or to a particular organ) by a given time or an indication that there is greater than a threshold (e.g., 80%) likelihood that the lesion 104 will metastasize by the given time.

In various cases, the subject 102 and the care provider 106 may be unaware of the presence of a second lesion of the subject 102, and the heterogeneity indicator 130 may indicate a likelihood of a presence or an absence of the second lesion. In some examples, the subject 102 presents with a second lesion. The heterogeneity indicator 130 includes one or more tumor heterogeneity conditions and/or prognostic features associated with the second lesion (e.g., the predicted presence or absence of tumor heterogeneity associated with the second lesion, at least one predicted cell type of the second lesion of the subject 102, a predicted tumor evolution of the second lesion of the subject 102, a predicted tumor progression of the second lesion of the subject 102, etc.). In some examples, the heterogeneity indicator 130 indicates an intralesional heterogeneity (e.g., genomic heterogeneity and/or phenotypic heterogeneity within the lesion 104 and/or within the second lesion) and/or interlesional heterogeneity (e.g., genomic heterogeneity and/or phenotypic heterogeneity between the lesion 104 and the second lesion). For instance, the heterogeneity indicator 130 may indicate one or more differences between the at least one predicted cell type of the lesion 104 and the at least one predicted cell type of the second lesion.

In various examples, the prognostic features include a predicted condition (e.g., disease) of the subject 102; a predicted disease subtype of the subject 102; a predicted survivability of the subject 102; one or more predicted symptoms of the subject 102; a predicted (e.g., suggested) effective therapy to treat the predicted disease of the subject 102; a dosage of one or more therapeutic agents (e.g., biologics, chemotherapeutic agents, etc.) predicted to treat the condition of the subject 102, a predicted stage of the predicted disease of the subject 102; a predicted grade of the predicted disease of the subject 102; a predicted activity level of the subject 102 (e.g., a predicted Eastern Cooperative Oncology Group (ECOG) performance status of the subject 102); a predicted diabetes status of the subject 102; a predicted body mass index (BMI) of the subject 102; a predicted smoking history of the subject 102; a predicted breast density of the subject 102; a clinical trial that the subject 102 is predicted to qualify (e.g., be eligible) for; or a characteristic of the predicted disease of the subject. For instance, the heterogeneity indicator 130 may indicate that the subject 102 is likely to qualify for a clinical trial based on an age, a gender, a disease stage, and previous treatments of the subject 102.

In some examples, the heterogeneity indicator 130 may indicate one or more cell populations in the tumor of the subject. For instance, the heterogeneity indicator 130 may be indicative of a number of cell population(s), a proportion of each of the cell population(s), a primary (e.g., most prevalent) cell population, a secondary (e.g., 2nd most prevalent) cell population, characterizations of the cell population(s), a tumor evolution, one or more cancer cell types in the tumor, one or more cancer cell subtypes in the tumor, a mutational profile, alteration-level clonality, or a cell type profile of the tumor. In some examples, the heterogeneity indicator 130 may indicate a presence of germline variants in cells of the tumor or a presence of somatic variants in cells of the tumor. Accordingly, the condition of the subject 102 can be determined based on the input features 126.

In some implementations, the predictive model 128 is unable to conclusively categorize the tumor heterogeneity of the subject 102. For example, the predictive model 128 may determine that the input features 126 of the subject 102 do not fit within any of the previously defined clusters in the clustering model. In various cases, the predictive model 128 may output an indication that that the categorization of the tumor heterogeneity is inconclusive.

A report generator 132 is configured to generate a report 134 based, at least in part, on the heterogeneity indicator 130. The report 134, for example, includes consumable data that can inform the care provider 106 about the predicted condition of the subject 102. In various implementations, the report 134 may indicate the results of additional analyses, such as the results of a histological study, whole transcriptome sequencing, cfRNA sequencing, whole exome sequencing, whole genome sequencing, a cancer (e.g., DNA) hotspot panel test, a DNA methylation test, a TMB test, a DNA fragmentation test, an RNA fragmentation test, a microsatellite instability (MSI) test, or a viral status test. The performance of such tests is within the ordinary skill of the art, with additional detail provided elsewhere herein. The report 134, for example, may include a genomic profile of the subject 102 based on various combinations of the above analyses and tests.

In some implementations, the report 134 indicates that a follow-up test of the subject 102 is indicated. For instance, in response to determining that the categorization of the tumor heterogeneity of the subject 102 is inconclusive, the report generator 132 may generate the report 134 to indicate that one or more additional tests, such as a physical exam, a histological study, or a nucleic acid sequencing-based test (e.g., genome sequencing, exome sequencing, additional DNA sequencing, RNA sequencing, transcriptome sequencing, etc.) should be performed in order to accurately identify the tumor heterogeneity of the subject 102. In some examples, the one or more addition tests may include diagnostic imaging, such as magnetic resonance imaging, computed tomography scan, ultrasound, X-ray, mammogram, positron emission tomography, bone scintigraphy, myelography, virtual colonoscopy, echocardiography, radiography, nuclear medicine, fluoroscopy, or single-photon emission computed tomography.

In various cases, the report 134 is output to a clinical device 136. For example, the report generator 132 transmits the report 134 to the clinical device 136. In various implementations, the clinical device 136 is a computing device that is operated by, owned by, or otherwise associated with the care provider 106. For instance, the clinical device 136 may be a desktop computer, a laptop computer, a smart phone, or some other computing device associated with the care provider 106. The clinical device 136, in various cases, outputs the report 134 to the care provider 106. In some cases, the clinical device 136 includes a display (e.g., a screen) that visually presents the report 134. In various cases, the clinical device 136 includes a speaker that outputs a sound indicative of the report 134. The clinical device 136, in various cases, may output the information in the report 134 using one or more output mechanisms or devices.

The care provider 106 may review the report 134 by interacting with the clinical device 136. The report 134, in various cases, may enhance the clinical decision-making of the care provider 106. For instance, the care provider 106 may determine a therapy for the subject 102 based on the report 134, such as drug therapy, radiation therapy, targeted therapy, vaccine therapy, stem cell transplantation, blood transfusion, physical therapy, psychiatric therapy, or surgery. The care provider 106 may prepare and/or administer a therapy to the subject 102 based on the report 134. According to various implementations, the care provider 106 may initiate the therapy and/or refer the subject 102 to another care provider to receive the therapy. In various cases, if the predicted condition of the subject 102 is a disease (e.g., cancer), the care provider 106 may prescribe, recommend, or administer an agent in order to treat the disease the subject 102.

In various implementations, the care provider 106 may develop a diagnosis and/or prognosis of the subject 102 based on the report 134. In various implementations, the care provider 106 may communicate information in the report 134 to the subject 102.

FIG. 1 illustrates various elements that can be embodied in one or more computing devices. For example, at least a portion of the functions of one or more of the sequencer 112, the preprocessor 116 the data transformer 120, the feature selector 124, the predictive model 128, the report generator 132, or the clinical device 136 are performed by one or more processors in at least one computing device. Examples of computing devices include server computers, desktop computers, laptop computers, tablet computers, mobile phones, wearable devices, Internet of Things (IoT) devices, and the like. In various cases, instructions for performing at least a portion of the functions of these elements are stored in memory and/or in a non-transitory computer readable medium. The instructions, for instance, are executed by the processor(s).

FIG. 1 also illustrates various types of data. For example, one or more of the sequence read data 114, the preprocessed data 118, the transformed data 122, the input features 126, the heterogeneity indicator 130, or the report 134, or any combination thereof, includes data. The various types of data illustrated in FIG. 1 may be stored, such as in memory or in non-transitory computer readable media. In various implementations, at least a portion of the data is transmitted or otherwise output by one or more computing devices. For example, a computing device may transmit one or more communication signals to another computing device, wherein the communication signal(s) encode at least a portion of the data. Examples of communication signals include electromagnetic signals, optical signals, ultrasonic signals, optical signals, and electrical signals. For example, communication signals can be transmitted wirelessly and/or in a wired fashion. The communication signals, for instance, are transmitted over one or more wireless channels and/or one or more wired channels (e.g., optical cabling, electrical cabling, etc.). In various cases, the communication signal(s) are transmitted over one or more communication networks. A communication network, for instance, may be defined according to one or more physical channels, such as one or more frequency spectra. In some cases, a communication network is defined according to one or more communication protocols and/or standards. Examples of communication networks include fiber optic networks, Institute of Electrical and Electronics Engineers (IEEE) networks (e.g., WI-FI™ networks, WiMAX networks, BLUETOOTH™ networks, etc.), cellular networks (e.g., a 3rd Generation Partnership Project (3GPP) radio network, such as a Long Term Evolution (LTE) network, a New Radio (NR) network; or a cellular core network such as a 3rd Generation (3G) core, a 4th Generation (4G) core, a 5th Generation (5G) core, etc.), ultrasonic networks, and the like. In some cases, the data is broadcasted from one device to multiple other devices. In some cases, the data is unicasted from one device to another device. For instance, various forms of data described herein may be transmitted via a peer-to-peer (P2P) connection.

A particular example will now be described with reference to FIG. 1. In this example, the subject 102 presents to a clinical environment for an assessment (e.g., a follow-up visit) of a lung cancer of the subject 102. The care provider 106 may, without ordering imaging of the subject 102, obtain the sample 108 from the blood of the subject 102. The sequencer 112 may generate sequence read data 114 based on DNA fragments within the blood sample 108 of the subject 102. For example, the sequence read data 114 may represent endpoint positions of the DNA fragments within one or more genes associated with a tumor heterogeneity. The preprocessor 116 may generate the preprocessed data 118 by normalizing and smoothing the sequence read data 114. In some examples, the data transformer 120 may generate the transformed data 122 by transforming the preprocessed data 118 into the frequency domain. The feature selector 124 may generate the input features 126 based on identifying attributes of the transformed data 122 that are indicative of tumor heterogeneity. The predictive model 128 may generate the heterogeneity indicator 130 based on the input features 126. The heterogeneity indicator 130 may indicate three cell populations associated with the lesion 104. In some examples, the heterogeneity indicator 130 may indicate that a dominant cell population (e.g., the most prevalent cell population) may be resistant to EGFR tyrosine kinase inhibitors (TKIs), and the non-dominant cell populations are susceptible to EGFR TKIs. The heterogeneity indicator 130 may indicate that the dominant cell population may be susceptible to chemotherapy or immunotherapy, such as pembrolizumab or nivolumab. In some examples, the heterogeneity indicator 130 may indicate a proportion of each of the three cell populations. In some examples, the heterogeneity indicator 130 is indicative of a predicted tumor progression of the lesion 104, a predicted survivability of the subject 102, a tumor evolution of the lesion 104, a predicted tumor evolution of the lesion 104, or a predicted metastasis profile of the lesion 104. For instance, the predictive model 128 may determine that the lesion 104 will not metastasize in the next 6 months because the tumor heterogeneity is not associated with aggressive progression. In some cases, the predictive model 128 outputs the heterogeneity indicator 130 based on a more sophisticated analysis of various characteristics of the input features 126, the transformed data 122, the preprocessed data 118, or the sequence read data 114.

Accordingly, the report generator 132 may generate the report 134 to indicate a recommendation to administer a combination therapy of a chemotherapy and a targeted therapy (e.g., an EGFR TKI) to the subject 102. Upon reviewing the report 134 on the clinical device 136, the care provider(s) 106, in some cases, administers the combination treatment to the subject 102. Accordingly, the rate of growth of the lesion 104 may stabilize or reduce due to treatment of all cell populations in the lesion 104.

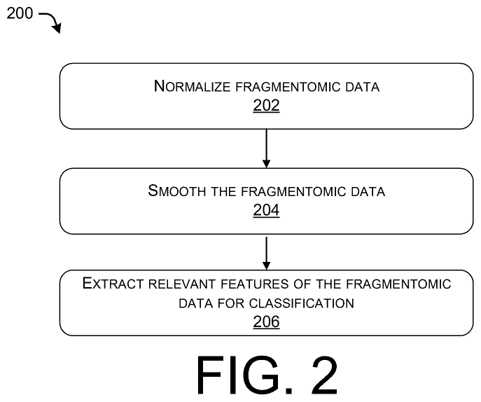

FIG. 2 illustrates example process 200 for preprocessing fragmentomic data for use in tumor heterogeneity classification. Different biological states, including tumor types, cell types, blood types, biomarkers, and the like, produce different patterns of fragmentation in biological patterns. However, raw endpoint density and other types of fragmentomic data can be impacted not only by the nucleic acid fragments in the sample being processed, but also by sources of artifact. These sources, for instance, include discrepancies due to low tumor fraction in the sample, sequencing errors, sequencing frequency due to bait molecule genomic location, and shearing of fragments during sample acquisition and processing. Due to the presence of these artifacts, it may be difficult to infer biologically relevant fragmentomic patterns in raw fragmentomic data.

Various implementations of the present disclosure address these and other challenges by preprocessing fragmentomic data before analysis. Example techniques described herein can remove artifact from fragmentomic data. According to various cases, preprocessing techniques described herein can enhance the accuracy, sensitivity, and specificity of various classifications performed using fragmentomic data. For instance, techniques described herein can enhance the accuracy of identifying a tumor heterogeneity of a subject based on fragmentomic data generated based on one or more samples obtained from the subject. Techniques described herein are particularly relevant for screening techniques, wherein a sample with a relatively small amount of relevant fragments can be used to accurately assess whether the subject has the tumor heterogeneity.

At 202, coverage of fragmentomic data is normalized. Various sequencing techniques described herein result in different portions of a region being sequenced at different amounts or rates. In particular cases, sequences that correspond to target regions used to generate the fragmentomic data are sequenced at a higher rate than other sequences. Various bait molecules, for example, are selected within the target region (e.g., a gene or other subgenomic interval-of-interest) in order to enhance the amount of signal obtained in the target region during sequencing. For instance, the sequences that correspond to the bait molecules are tiled (e.g., arranged, with or without interspersed gaps) across the target region. In various cases, the raw fragmentomic data is normalized based on sequence read data that corresponds to bait molecules used to generate the fragmentomic data. For example, an average endpoint count across a bait molecule sequence or the target sequence is calculated, and the remaining endpoint count data is normalized based on that average.

At 204, the fragmentomic data is smoothed. In various cases, patterns of fragmentomic data that are relevant to classification are not necessarily apparent at the single-base level. Therefore, smoothing the fragmentomic data can enhance the signal-to-noise ratio of the fragmentomic data without removing potentially relevant fragmentomic features. According to various implementations, the endpoint count for a given position in the smoothed fragmentomic data is assigned as an average (e.g., a mean, a median, etc.) endpoint count for a window of genomic positions in the fragmentomic data. The window of genomic positions, for example, is symmetric at the position. In various cases, the width of the window is in a range of ±5 to ±50 genomic positions around the position. For example, the width of the window is ±5, ±10, ±15, ±30, or ±50 genomic positions around the position. In some cases, the position is assigned as a weighted average of the endpoint counts within the window. For example, the smoothed endpoint counts can be generated by convolving, cross-correlating, or multiplying a two-dimensional kernel (e.g., a Gaussian filter) with the endpoint counts in the pre-smoothed fragmentomic data, wherein the two-dimensional kernel itself has the width in the range of ±5 to ±50 genomic positions. Accordingly, in some cases, the smoothed endpoint count at a given position is more dependent on endpoint counts in the center of the window compared to endpoint counts at the edge of the window.

At 206, relevant features of the fragmentomic data are extracted for classification. In some cases, the features include and/or are based on the entire set of fragmentomic data. In some examples, the relevant features include and/or are based on a subset of the fragmentomic data. For instance, the preprocessed data 118 described above with reference to FIG. 1 includes the relevant features generated at 206.

According to some cases, the fragmentomic data is further processed if the sample itself has been classified as a low-signal sample. For instance, this additional processing step can be selectively performed for samples that are determined to have less than a threshold amount of fragments that have originated from cells relevant to the classification. In the case of tumor classification, this processing step can be performed on fragmentomic data derived from samples having less than a threshold tumor fraction. According to various cases, baseline fragmentomic data is generated based on multiple low-signal samples derived from a population that omits the subject. The baseline fragmentomic data, for instance, includes the average (e.g., mean) endpoint count in the low-signal samples and/or the standard deviation of the endpoint counts in the low-signal samples at each genomic position in the target region.

In various cases, the baseline fragmentomic data is compared to the (e.g., normalized and/or smoothed) fragmentomic data of the sample. A statistic is calculated for each genomic position based on the comparison of the baseline fragmentomic data and the fragmentomic data of the sample. That is, the fragmentomic data of the sample is transformed into an alternate space. The statistic, for example, represents an amount of a discrepancy between the fragmentomic data of the sample as compared to the baseline fragmentomic data. For instance, a Z-score, a t-statistic, p-value, or other type of statistic is generated for each genomic position. The Z-score, for instance, represents the number of standard deviations by which the endpoint count in the fragmentomic data of the sample deviates from the average endpoint count in the low-signal samples. The fragmentomic data of the sample, for instance, is transformed into a Z-score space. In various implementations, the genomic positions corresponding to a statistic value (e.g., a Z-score) that outside of a threshold range (e.g., a confidence interval) are preferentially relied upon for classification. These genomic positions, for instance, identify whether the fragmentomic data of the sample is abnormal. In various cases, the features of the fragmentomic data that are extracted for classification include, or are derived from, the portions of the fragmentomic data that have statistic values outside of the threshold range. In various implementations, data derived from genomic positions having statistic values (e.g., Z-scores) that are within the threshold range (e.g., the confidence interval) are omitted from the fragmentomic features used for classification. Thus, the comparison between the baseline fragmentomic data and the fragmentomic data of the sample can be used to differentiate portions of the fragmentomic data of the sample that are relevant or irrelevant to determining whether the subject has the tumor heterogeneity. The comparison, for instance, can be utilized to reduce the background signal of the fragmentomic data of the sample in order to enhance and simplify a subsequent classification process.

According to some cases, the relevant features extracted from the preprocessed fragmentomic data are used to identify whether the subject has the tumor heterogeneity. In various examples, the relevant features include, or are based on, portions of the preprocessed fragmentomic data that are converted into an alternate domain. In some cases, the relevant features are input into an ML model that is configured to classify the sample as having the condition or lacking the tumor heterogeneity. For example, the ML model is supervised or unsupervised.

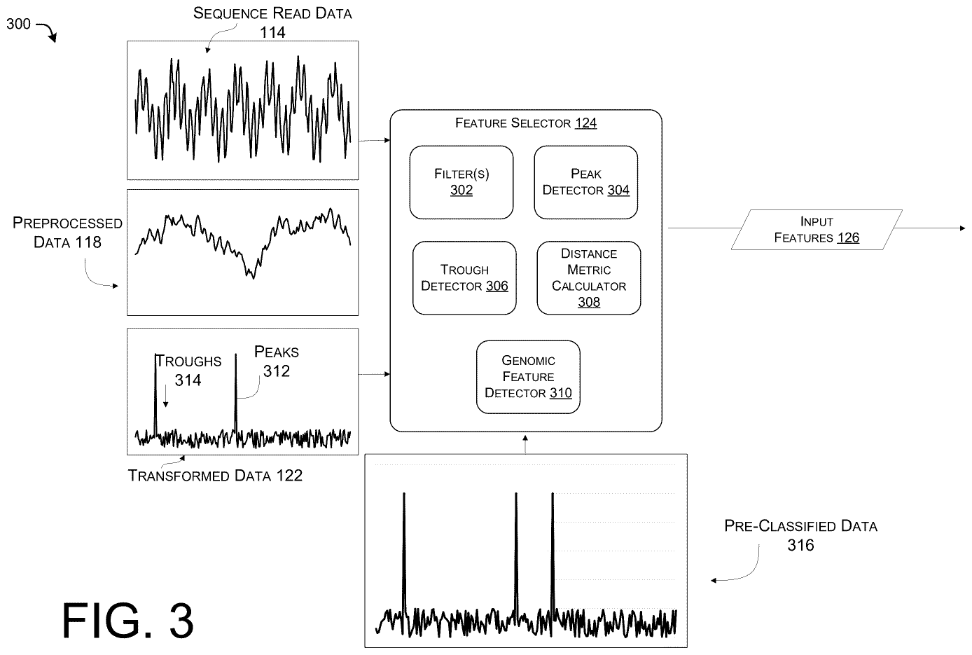

FIG. 3 illustrates example signaling 300 for selecting features for classifying a tumor heterogeneity of a subject based on transformed genomic information of the subject. The signaling 300 is to and from the feature selector 124 described above with reference to FIG. 1, for instance. The signaling 300 further includes the sequence read data 114, the preprocessed data 118, the transformed data 122, and the input features 126 described above with reference to FIG. 1.

The sequence read data 114 represents sequences of nucleic acid molecules in a sample obtained from a subject. In some examples, the sequence read data 114 is multi-dimensional data. One of the dimensions of the sequence read data 114, for instance, represents genomic position. In some examples, one of the dimensions of the sequence read data 114 represents a number of endpoints (e.g., a number of right endpoints and/or left endpoints, also referred to as “endpoint counts”) of fragments in the nucleic acid molecules detected in the sample. In some examples, the dimensions of the sequence read data 114 include at least one of a presence (or absence) of variants in the nucleic acid molecules, an amount of signal observed by a sequencer (e.g., at a given genomic position) from the nucleic acid molecules, a read depth, a length of fragments in the nucleic acid molecules, or any combination thereof. The sequence read data 114, for instance, represents the sequences of the nucleic acid molecules in a spatial domain that is defined by genomic position. In some cases, the sequence read data 114 represents genomic positions in at least one locus. For instance, the sequence read data 114 may be limited to genomic positions in one or more genes-of-interest that are relevant for classifying the condition of the subject.

In various cases, the preprocessed data 118 is also multi-dimensional. In some cases, the preprocessed data 118 is a normalized and/or smoothed version of the sequence read data 114, such that the preprocessed data 118 has a reduced level of noise compared to the sequence read data 114. In some implementations, the preprocessed data 118 is in the form of a frequency distribution of endpoint counts of fragments in the nucleic acid molecules.

Similar to the sequence read data 114 and the preprocessed data 118, the transformed data 122 is multi-dimensional and also represents the sequences of the nucleic acid molecules in the sample obtained from the subject. However, the transformed data 122 may be mapped to an alternate domain compared to the spatial domain of the sequence read data 114. For instance, a dimension of the sequence read data 114 may be a frequency domain rather than a spatial domain. The transformed data 122 may be generated by performing at least one transform on the sequence read data 114. Examples of transforms include a Fourier transform, a Laplace transform, a Mellin transform, a wavelet transform (e.g., a continuous wavelet transform (CWT), a discrete wavelet transform (DWT), a fast wavelet transform (FWT), a complex wavelet transform, a Newland transform, a stationary wavelet transform (SWT), a second generation wavelet transform (SGWT), a dual-tree complex wavelet transform (DTCWT), etc.), or any combination thereof.

In various cases, the feature selector 124 generates the input features 126 based on the sequence read data 114, the preprocessed data 118, and the transformed data 122. The input features 126, for instance, include characteristics of the subject that are relevant to determining a condition of the subject, and which are derived based on the sequence read data 114, the preprocessed data 118, the transformed data 122, or any combination thereof.

In some examples, the feature selector 124 includes at least one filter 302 configured to remove and/or enhance characteristics of the sequence read data 114, the preprocessed data 118, the transformed data 122, or any combination thereof. In particular cases, the filter(s) 302 is configured to remove an artifact of the sequence read data 114 and/or the transformed data 122. Examples of filters that can be included in the filter(s) 302 include at least one of a Butterworth filter, a Chebyshev filter, an FIR filter, an IIR filter, a low-pass filter, a high-pass filter, or a bandpass filter. In some cases, the filter(s) 302 is a set of data having a shape that is suitable for removing and/or enhancing characteristics of the sequence read data 114, the preprocessed data 118, the transformed data 122, or any combination thereof. The filter(s) 302, for instance, is multiplied, convolved, or cross-correlated with the sequence read data 114, the preprocessed data 118, the transformed data 122, or any combination thereof. In some cases in which the sequence read data 114 and preprocessed data 118 are in a spatial domain and the transformed data 122 is in a frequency domain, the filter(s) 302 is convolved with the sequence read data 114 and/or the preprocessed data 118, but is multiplied with the transformed data 122. In some cases, the filter(s) 302 is applied to the transformed data 122, and a reverse transform is performed on the filtered transformed data 122 in order to obtain filtered sequence read data 114 or filtered preprocessed data 118. According to some examples, the filtered sequence read data 114 and/or filtered preprocessed data 118 is utilized to perform various functions described herein.

In various cases in which the transformed data 122 is in a frequency (or frequency-related) domain, the transformed data 122 may include low-frequency and/or high-frequency artifact. Examples of low-frequency artifact include copy number deletions and/or copy number amplifications, when those features have limited to no relevance to the condition of the subject that is being assessed. In some cases, the sequencing technique used to generate the sequence read data 114 utilizes bait molecules associated with particular genomic regions (e.g., loci) of interest. Due to the physical limitations of this sequencing technique, there may an observed signal decay in genomic positions within a threshold of the bait molecules and/or at edges of the genomic regions of interest. This signal decay is another example of potential low-frequency artifact. In some examples, the filter(s) 302 includes a band-pass and/or a high-pass filter with a cutoff frequency that is suitable for removing one or more types of low-frequency artifact. In some cases, the sequence read data 114 further includes one or more types of high-frequency artifact. For example, the high-frequency artifact may include misreads during sequencing, base-level sequencing errors, alignment errors, or any combination thereof. The filter(s) 302, for instance, include a band-pass and/or low-pass filter with a cutoff frequency that is suitable for removing one or more types of high-frequency artifact.

In various cases, the input features 126 include the filtered sequence read data 114, the filtered preprocessed data 118, the filtered transformed data 122, or any combination thereof. In some examples, the input features 126 include one or more images representing the filtered sequence read data 114, the filtered preprocessed data 118, the filtered transformed data 122, or any combination thereof. According to some cases, the input features 126 include one or more features derived based on the filtered sequence read data 114, the filtered preprocessed data 118, the filtered transformed data 122, or any combination thereof. For example, the feature selector 124 may include a peak detector 304, a trough detector 306, a distance metric calculator 308, a genomic feature detector 310, or any combination thereof, configured to generate at least a portion of the input features 126 based on the filtered sequence read data 114, the filtered preprocessed data 118, and/or the filtered transformed data 122. Unless contradicted by context, it should be understood that any mention of the sequence read data 114, the preprocessed data 118, or the transformed data 122 may referred to unfiltered and/or filtered versions.

The peak detector 304, in various cases, is configured to detect peaks 312 in the data represented by the sequence read data 114, the preprocessed data 118, and/or the transformed data 122. Various types of peak detection methods can be utilized by the peak detector 304. For example, the peak detector 304 may identify the peaks by detecting all datapoints in a dataset that exceed a threshold (e.g., 50% of a maximum value of the dataset) and/or are larger than their respective neighboring datapoints. According to some cases, the peaks 312 identified by the peak detector 304 are indicated in the input features 126. For instance, the input features 126 may include a genomic position or other characteristic of the peaks 312 identified by the peak detector 304.

The trough detector 306, in various examples, is configured to detect troughs 314 in the data represented by the sequence read data 114 and/or the transformed data 122. Various types of trough detection methods can be utilized by the trough detector 306. For instance, the trough detector 306 may identify continuous segments of the sequence read data 114 and/or the transformed data 122 that are lower than a particular threshold (e.g., 35% of a maximum value of the dataset). The troughs 314 may be indicated in the input features 126. For example, the input features 126 may include a genomic position, start position, end position, or other characteristic of the troughs 314 identified by the trough detector 306.

In various cases, the distance metric calculator 308 is configured to compare the sequence read data 114 and/or the transformed data 122 with pre-classified data 316. The pre-classified data 316 may represent nucleic acid molecules obtained from another individual (e.g., not the subject) with a known condition (e.g., a known tumor heterogeneity condition). For instance, the pre-classified data 316 may be based on a sample obtained from an individual with a known tumor heterogeneity condition (e.g., a tumor including a single cell type). According to various cases, the pre-classified data 316 is in the same dimension as the sequence read data 114 and/or the transformed data 122.

According to various implementations, the distance metric calculator 308 is configured to generate a distance metric representing a similarity between the sequence read data 114 and the pre-classified data 316 and/or between the transformed data 122 and the pre-classified data 316. In some cases, the distance metric is low (e.g., close to 0) when the datasets are dissimilar, and high (e.g., approaching 1) when the datasets are similar. Various types of distance metrics are calculated by the distance metric calculator 308, such as a chi-squared distance, a Jensen-Shannon divergence, a Jaccard index, a Sorensen-Dice coefficient, or any combination thereof. In some cases, the datasets are convolved or cross-correlated together, and an area under the curve (AUC) or maximum of the resultant dataset is utilized as a distance metric.

In various cases, the distance metric calculator 308 is configured to generate the distance metric based on images of the datasets (e.g., an image of the transformed data 122 and an image of the pre-classified data 316). In some examples, the distance metric calculator 308 is configured to perform one or more image recognition techniques to identify the similarity between the datasets based on the images. For example, an image of the pre-classified data 316 may be one of a set of eigenimages generated by performing principal component analysis (PCA) on multiple images depicting sequence read data, preprocessed data, and/or transformed data from a population of multiple individuals. The image of the dataset to be classified (e.g., the image of the transformed data 122) is compared to the set of eigenimages to generate a set of weights (e.g., vectors generated by projecting the image on the set of eigenimages). The weights, for instance, may be included in the input features 126. In some cases, a distance metric (e.g., a Hamming distance, a Euclidian distance, or the like) representing a similarity between the weights of the image to be classified and weights representing projections of the pre-classified data 316 on the eigenimages is included in the input features 126. In some cases, images of the sequence read data 114, the preprocessed data 118, and/or the transformed data 122 are included in the input features 126.

The genomic feature detector 310 is configured to determine one or more genomic features of the subject by analyzing the sequence read data 114, the preprocessed data 118, and/or the transformed data 122. For example, the genomic feature detector 310 may calculate at least one of a mutational profile of the sample, a mutational signature of the sample, an MMRD probability score, a copy number state, a fraction unstable score, or the presence of one or more pathogenic variants by analyzing the sequence read data 114, the preprocessed data 118, and/or the transformed data 122. One or more of the genomic features may be included in the input features 126.

In various implementations, the input features 126 are utilized to identify a condition of the subject. For example, the input features 126 are provided to a classifier configured to predict whether the subject has one or more conditions, or does not have the one or more conditions. According to various cases, the classifier is configured to determine whether the subject has a particular tumor heterogeneity condition (e.g., a homogenous tumor, a tumor with two distinct cell types, etc.). In some cases, the classifier includes one or more ML models.

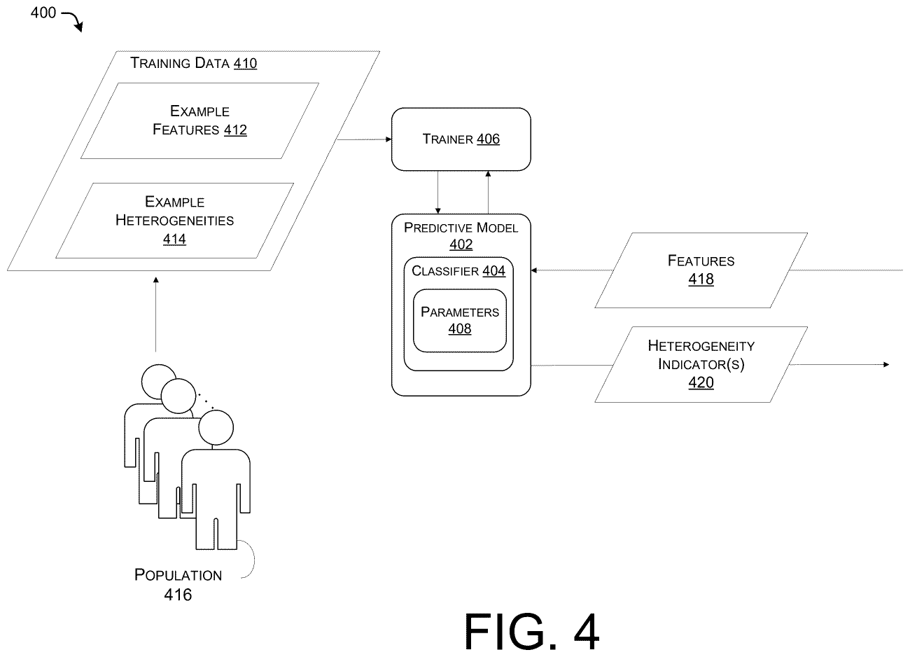

FIG. 4 illustrates an example environment 400 for training and utilizing a predictive model 402 to identify a tumor heterogeneity of a subject. The predictive model 402, for instance, is the predictive model 128 described above with reference to FIG. 1. In various implementations, the predictive model 402 includes a classifier 404, which may include one or more ML models. A trainer 406, for instance, is configured to optimize various parameters 408 of the classifier 404 based on training data 410.

The training data 410 includes example features 412 and example heterogeneities 414. The example features 412, in various cases, are obtained based on nucleic acid molecules of individuals within a population 416. In various examples, the example features 412 include, or are derived, based on preprocessing and/or transforming sequence read data of the nucleic acid molecules into an alternate domain (e.g., transformations of the sequence read data from a spatial domain to a frequency or wavelet domain). In some cases, the example features 412 include fragmentomic features of the population 416. The example heterogeneities 414 may include indications of tumor heterogeneities of the individuals within the population 416. For example, the example heterogeneities 414 may include indications of whether the individuals within the population 416 have a particular tumor heterogeneity condition (e.g., tumors with a single cell type, tumors with multiple cell types, etc.). In some cases, the example heterogeneities 414 may be generated based on clinical evaluations and/or nucleic acid sequencing-based tests of the individuals within the population 416, such as by one or more care providers.

The classifier 404 includes one or more model types. For instance, the classifier 404 includes an artificial neural network. An artificial neural network includes various layers that respectively process input data. For example, an artificial neural network includes an input layer, one or more hidden layers, and an output layer. The input layer performs a preprocessing operation on the input data. The hidden layer(s) may perform various processing operations on the output from the input layer. The output layer, in various cases, processes the output from the hidden layer(s). Each layer, in some cases, includes one or more nodes, which are defined by individual operations. In various cases, the hidden layer(s) include nodes that are connected to each other in parallel and/or series. Examples of artificial neural networks include feedforward neural networks, multi-layer perceptrons (MLPs), convolutional neural networks (CNNs), and backpropagation models. In various implementations, the operations performed by the layers and/or nodes within an artificial neural network included in the classifier 404 is defined according to the parameters 408. For example, the parameters 408 may include weights, thresholds, filters, kernels, or other data objects that are utilized to perform operations of the classifier 404.

In some implementations, the classifier 404 includes a nearest-neighbor model. One example of a nearest-neighbor model includes a k-nearest neighbor model. For example, a nearest-neighbor model defines various “neighbors,” which are points within a feature space, with associated class labels. When a new data point is mapped to the feature space, the new data point is classified based on the proximity (e.g., Euclidian distance, Manhattan distance, Minkowski distance, etc.) of its “neighbors” to the new data point as well as their associated classes. In some cases, the new data point is classified as belonging to a particular class if greater than a threshold number of neighbors within a threshold distance of the new data point are members of the class. For instance, the parameters 408 may include k (e.g., the number of neighbors compared to the new data point), the threshold distance, and so on.

In various cases, the classifier 404 includes a regression analysis model. The regression analysis model, for example, is defined by a regression function that defines relationships between one or more independent variables and one or more dependent variables. The regression function may further define one or more unknown parameters that define a relationship between the independent and dependent variables. In various implementations, the unknown parameters and/or the type of regression function (e.g., linear, quadratic, etc.), is defined according to the parameters 408.

In some cases, the classifier 404 includes a clustering model. In various cases, a clustering model maps various data points (e.g., training data) to a feature space. Based on the proximity of groups of those data points in the features pace, one or more “clusters” are defined. An additional data point may be classified according to one or more of the clusters based on its proximity to the clusters (e.g., a center of the clusters, a boundary of the cluster, etc.). Examples of clustering models include k-means clustering, mean-shift clustering, expectation-maximization (EM) clustering, and agglomerative hierarchical clustering. The parameter(s) 408, for example, include a threshold proximity within which a new data point is classified within a cluster, a density of points used to define a cluster, and the like.

In various examples, the classifier 404 includes a principal component analysis model. In various implementations, a principal component analysis defines a collection of principal components of unit vectors within a coordinate space based on a data set (e.g., training data). The model, for example, is an orthogonal linear transformation of the data set. Various weights of the model, for example, are included in the parameter(s) 408.

The classifier 404, in some implementations, includes a gradient boosting model. For example, the gradient boosting model is defined as a collection of prediction models (e.g., decision trees) that iteratively classify observed data. In various cases, the type of prediction model, weights in the prediction models, and the like, are defined by the parameter(s) 408.

The classifier 404, for example, includes a random forest. The random forest, for instance, includes multiple decision trees that classify data in an ensemble fashion. In various implementations, the decision trees are defined by the parameter(s) 408.

In various implementations, the classifier 404 includes a support vector machine (SVM). For example, the SVM includes a distribution of training samples (e.g., derived from the training data 410) in a multidimensional feature space. The SVM further includes a hyperplane that divides different classes of the training samples into different subspaces within the feature space, wherein each subspace corresponds to a different classification. In various cases, a new set of input features is classified by adding the input features to the feature space and determining the relative position of the input features to the hyperplane. In some cases, an SVM includes multiple hyperplanes. In various implementations, the training samples, classifications, and/or hyperplane(s) are defined by the parameter(s) 408.

In some cases, the classifier 404 includes a probabilistic classifier, such as a naëve Bayes classifier. In some cases, a naëve Bayes classifier is generated based on average (e.g., mean) values and variances of features (e.g., fragmentomic features) for each class (e.g., tumor heterogeneity condition) in training samples. The features are assumed, in some cases, to have a particular distribution (e.g., Gaussian distribution) among the population of training samples. In various cases, a new set of input features is classified by calculating the probability that the input features fit each class defined in the classifier. In various implementations, the average values, variances, distributions, and other characteristics of the classifier are defined by the parameter(s) 408.

In various implementations of the present disclosure, the trainer 406 is configured to optimize the parameters 408 based on the training data 410. For example, the trainer 406 may input first example features (corresponding to a first individual among the population 416) among the example features 412 into the predictive model 402 and may receive a predicted heterogeneity condition of the first individual as a result of computations performed using the predictive model 402. The trainer 406 may compute a loss (e.g., determine a discrepancy) between a first example heterogeneity condition (corresponding to the first individual) among the example heterogeneities 414 and the predicted heterogeneity condition. Further, the trainer 406 may alter the parameters 408 in order to minimize the loss. In various cases, the trainer 406 optimizes the parameters 408 iteratively based on the entire set of the training data 410.

In various implementations, the optimization of the parameters 408 enables the predictive model 402 to identify predictive attributes of the example features 412 that are correlated to or otherwise associated with the example heterogeneities 414. For instance, the predictive model 402 may determine that a particular peak pattern represented in transformed data among the example features 412 is highly correlated with adenosarcoma. The predictive model 402 may therefore classify heterogeneity conditions (e.g., one or more cancer cell types and/or cancer cell subtypes) based on features outside of the example features 412 by recognizing or otherwise identifying the predictive attributes.

Once the parameters 408 are optimized, the predictive model 402 may be ready to classify a new set of data. For example, the predictive model 402 may receive input data including features 418 of a subject. The features 418, for instance, may include one or more of the predictive attributes that are relevant for classifying a heterogeneity condition of the subject. According to various implementations, the features 418 are based on transforming sequence read data of the subject into the alternate domain. In various cases, the features 418 include fragmentomic features. The predictive model 402 may perform various operations on the input data based on the trained classifier 404 and the optimized parameters 408. In various cases, the predictive model 402 outputs output data including one or more heterogeneity indicators 420 based on the features 418. The heterogeneity indicator(s) 420, for instance, include one or more predicted tumor heterogeneities (e.g., a predicted tumor evolution, a predicted metastasis profile, etc.) of a cancer experienced by the subject.

Although FIG. 4 is primarily described as referring to supervised learning, implementations are not so limited. In various cases, the training data 410 omits the example heterogeneities 414 and the trainer 406 is configured to optimize the parameters 408 using the example features 412 and an unsupervised learning technique.

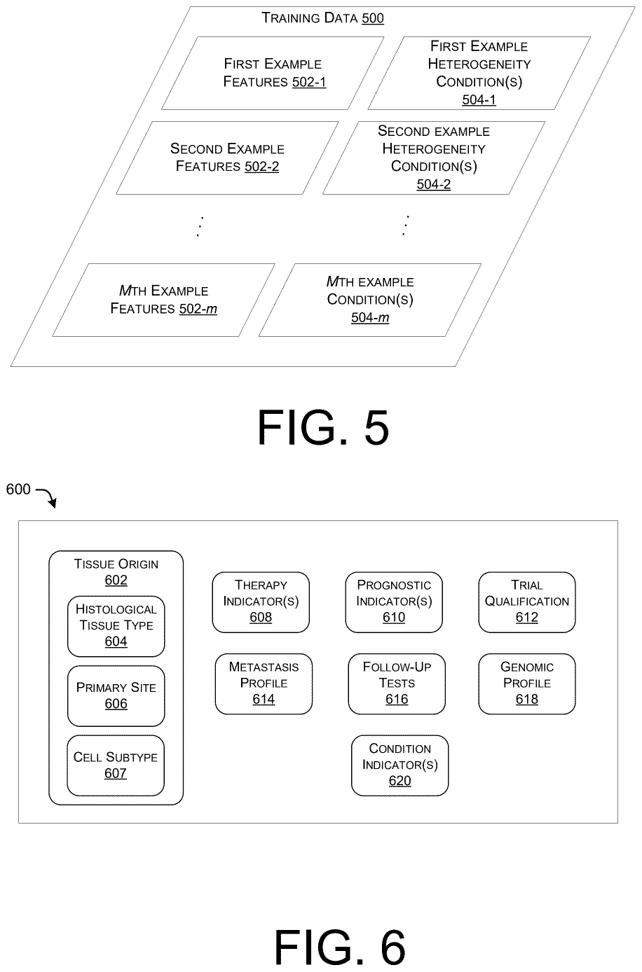

FIG. 5 illustrates an example of training data 500 utilized to train one or more ML models. For example, the training data 500 may be the pre-classified data 316 described above with reference to FIG. 3.

The training data 500, in various cases, may represent m samples, wherein m is a positive integer. In some cases, the m samples are respectively obtained from m individuals within a population, although implementations are not so limited. For example, in some cases, multiple samples may be obtained from the same individual at different times.

The training data 500 includes first to mth example features 502-1 to 502-m. For example, the first to mth example features 502-1 to 502-m include features derived from nucleic acid molecules in the respective m samples. In some cases, spatial domain data is obtained by sequencing the nucleic acid molecules. According to various implementations, the spatial domain data is converted to an alternate domain (e.g., a frequency or wavelet domain) to generate the first to mth example features 502-1 to 502-m. In various cases, the first to mth example features 502-1 to 502-m include fragmentomic features.

The training data 500 may further include first to mth example heterogeneity conditions 504-1 to 504-m. The first to mth example heterogeneity conditions 504-1 to 504-m, for instance, include heterogeneity conditions of the individuals from which the m samples are obtained.

FIG. 6 illustrates an example report 600 summarizing predicted conditions (e.g., a tumor heterogeneity) of a subject. In various cases, the report 600 is the report 134 described above with reference to FIG. 1. The report 600, for instance, may be displayed to a patient and/or care provider. In some cases, the report 600 is generated based on features of a sample (e.g., a liquid biopsy sample) obtained from the subject. In various cases, the report 600 is generated based on fragmentomic features of the subject. In various cases, at least some elements of the report 600 are generated based on a predicted tumor heterogeneity of the subject.

In some cases, the subject is predicted to have a cancer. The report 600 includes a tissue origin 602 of the cancer. The tissue origin 602, for instance, indicates a histological tissue type 604, a primary site 606, cell subtype 607, or any combination, of the cancer.

In various cases, the report 600 includes one or more therapy indicators 608. For instance, the therapy indicator(s) 608 convey whether the cancer is predicted to be resistant to one or more predetermined therapies and/or whether the cancer is predicted to be responsive to one or more predetermined therapies.

In some examples, the report 600 includes one or more prognostic indicators 610. The prognostic indicator(s) 610, for instance, indicate a prognosis of the subject in view of the categorized cancer. For example, the prognostic indicator(s) 610 may indicate a survivability, a recoverability, a quality of life indicator, or other information indicative of the prognosis of the subject.

The report 600 may include a trial qualification 612 of the subject. The trial qualification 612, for instance, indicates whether the subject is predicted to qualify for a predetermined clinical trial.

The report 600, in various implementations, includes a metastasis profile 614 of the subject. The metastasis profile 614, for instance, indicates a likelihood that the cancer will metastasize (e.g., at a particular point in time), one or more tissues in which the cancer is predicted to metastasize, or the like.

In various cases, the report 600 includes recommended follow-up tests 616. For example, the report 600 may include a recommendation to perform whole genome sequencing on the subject, particularly in cases if the cancer cannot be categorized above a threshold certainty.

The report 600 may include a genomic profile 618 of the subject. In various cases, the genomic profile 618 includes or is generated based on the results of non-fragmentomic analyses of the subject.

In various implementations, the report 600 includes at least one condition indicator 620. The condition indicator(s) 620, for instance, indicate one or more predicted heterogeneity conditions of the subject. For instance, if the subject is predicted to have a type of cancer, the condition indicator(s) 620 may indicate a tumor heterogeneity associated with the tumor. Other types of conditions may also be noted in the condition indicator(s) 620, such as a predicted tumor evolution of the subject, a predicted metastasis profile of the subject, a predicted tumor progression of the subject, a predicted survivability of the subject, a general health of the subject, a genomic age of the subject, a risk that the subject will develop a disease, a predicted cancer of the subject, a predicted cancer subtype of the subject, a predicted effective therapy to treat the predicted pathology of the subject, a predicted stage of the predicted pathology of the subject, a predicted grade of the predicted pathology of the subject, an ECOG performance status of the subject. The condition indicator(s) 620 may indicate one or more cell populations in the tumor of the subject. For instance, the condition indicator(s) 620 may include a metric indicative of a number or a proportion of the cell population(s), characterizations of the cell population(s), one or more cancer cell types in the tumor, one or more cancer cell subtypes in the tumor, a mutational profile, alteration-level clonality, or a cell type profile of the tumor. In some examples, the condition indicator(s) 620 may indicate a presence of germline variants in cells of the tumor or a presence of somatic variants in cells of the tumor.

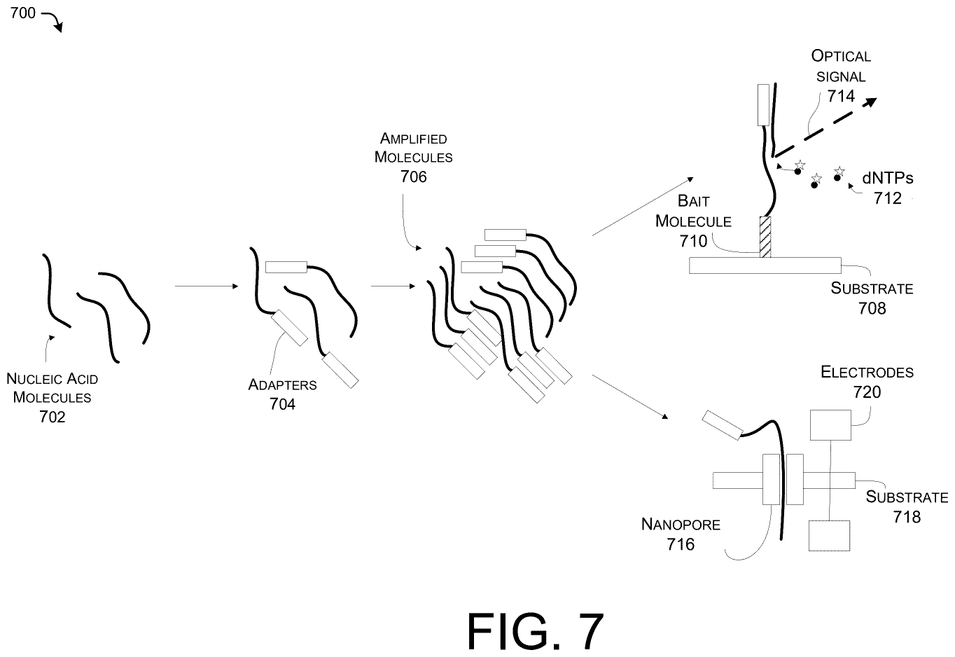

FIG. 7 illustrates an example environment 700 for sequencing various nucleic acid molecules 702. In various implementations, the nucleic acid molecules 702 include cfDNA and/or gDNA. For instance, the nucleic acid molecules 702 may include ctDNA. The nucleic acid molecules 702, in various cases, are extracted from a sample, such as a biological sample obtained from a subject. In some implementations, the nucleic acid molecules 702 include DNA that is complementary to RNA present in the sample.

The nucleic acid molecules 702, in various cases, are ligated with adapters 704. For examples, the adapters 704 are hybridized to the nucleic acid molecules 702. The adapters 704, for example, include additional nucleic acid molecules. In various implementations, the adapters 704 have a shorter length than the nucleic acid molecules 702 being sequenced. For instance, the adapters 704 include amplification primers, flow cell adapter sequences, substrate adapter sequences, or sample index sequences. Although FIG. 7 illustrates adapters 704 being ligated to one end of each of the nucleic acid molecules 702, implementations are not so limited. For example, the adapters 704 may be ligated to both ends of each of the nucleic acid molecules 702.

In various examples, the nucleic acid molecules 702 ligated with the adapters 704 are amplified in order to generate amplified molecules 706. Various amplification techniques can be performed. For instance, the amplified molecules 706 are generated using PCR, a non-PCR amplification technique, an isothermal amplification technique, or any combination thereof.

Amplified molecules 706 may be captured by bait molecules 710 and sequenced. In some implementations, the amplified molecules 706 are sequenced via sequencing-by-synthesis. In various cases, fluorescently tagged deoxyribonucleotide triphosphates (dNTP) 712 are utilized to synthesize a strand that is complementary to DNA strands bound to the substrate 708. When a dNTP 712 is added to the strand (e.g., by an enzyme), the dNTP 712 emits an optical signal 714. In various implementations, the frequency of the optical signal 714 is dependent on the type of dNTP 712 from which the optical signal 714 is emitted. By detecting the optical signals 714 as the strand is being synthesized, the sequence of the original nucleic acid molecules 702 can be derived.

In some implementations, the amplified molecules 706 are sequenced via nanopore sequencing. For instance, the amplified molecules 706 are directed through a nanopore 716 extending through a substrate 718. In various cases, the amplified molecules 706 are negatively charged, such that they can be directed through the nanopore 716 by imposing an electrical field across the substrate 718. In various cases, the amplified molecules 706 and the nanopore 716 are in the presence of a charged solution. Thus, charged solutes traveling through the nanopore 716 can be monitored by reviewing an electrical signal (e.g., a current) sensed between electrodes 720 on either side of the substrate 718. As an amplified molecule 706 is directed through the nanopore 716, the individual bases within the amplified molecule 706 will block the nanopore 716, which may decrease the amount of charged solutes traveling through the nanopore 716 and consequently, the magnitude of the electrical signal detected by the electrodes 720. Each of the four types of bases within the amplified molecules 706, may block the nanopore 716 to a different extent. Therefore, the sequences of the nucleic acid molecules 702 can be derived by analyzing the measured electrical signal with respect to time as the amplified molecules 706 are directed through the nanopore 716.

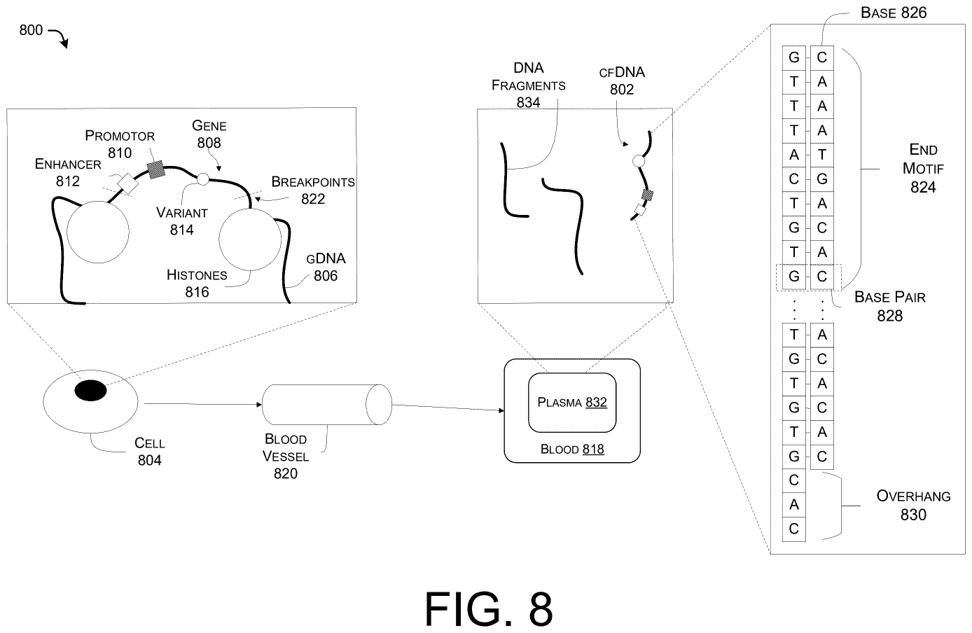

FIG. 8 illustrates an example environment 800 illustrating cfDNA 802, which can be utilized to a condition of a subject. For instance, the cfDNA 802 may be included in the nucleic acid molecules 110 described above with reference to FIG. 1.

In various implementations, a cell 804 within the subject includes genomic DNA (gDNA) that is expressed by the cell 804. In some cases, the cell 804 is a cancer cell. For example, the gDNA 806 may include various sequences, such as a gene 808, a promoter 810, an enhancer 812, and a variant 814. For example, the variant 814 is part of the gene 808. In addition, various epigenetic factors impact expression of the gene 808 as well as other genes within the gDNA 806. For example, the gDNA 806 may be packaged within the nucleus of the cell 804 with various histones 816. When the gene 808 is expressed, a portion of the gDNA 806 including the gene 808, the promotor 810, the enhancer 812, and the variant 814 may be exposed to proteins within the nucleus, such as RNA transcriptase. In various cases, the portion of the gDNA 806 is unwrapped or otherwise unpackaged from the histones 816. Thus, the expression of the gene 808 (e.g., the amount of mRNA generated by RNA transcriptase based on the gene 808 within the cell 804) is linked to the frequency or time at which the portion of the gDNA 806 is exposed.

The cell 804, for example, may die. The contents of the cell 804, including the gDNA 806, may be released. In various cases, the gDNA 806 is released into blood 818 that flows through a blood vessel 820 of the subject. When the gDNA 806 is released from the nucleus of the cell 804, the gDNA 806 is degraded due to various biophysical and/or biochemical factors. For example, the blood 818 may include various enzymes that cut the gDNA 806 into the cfDNA 802. In various cases, other mechanical, chemical, or thermal conditions in the blood 818 divide the gDNA 806 into the cfDNA 802. For example, these conditions divide the gDNA 806 into fragments at various breakpoints 822.

Notably, the presence and location of the histones 816 may impact the sequences of the cfDNA 802 that are observed in the blood 818. The breakpoints 822, for example, are more likely to occur at edges of a sequence of the gDNA 806 that is exposed by the histones 816. Therefore, the sequence of the cfDNA 802 is indicative of the expression of mRNA and other functional RNA in the cell 804. By reviewing the cfDNA 802, the expression of the cell 804 can be determined without performing RNA sequencing, in some cases. In various examples, the expression of the cell 804 is relevant to the condition of the subject.

In addition, the sequences at or near the breakpoints 822 are indicative of expression of the cell 804. For example, the cfDNA 802 may include an end motif 824. The end motif 824 may be defined as a sequence of bases 826 and/or base pairs 828 that extend from an end of the cfDNA 802. The end motif 824, for example, has a predetermined length that is in a range of 1 to 30 bases and/or base pairs. In various implementations, the cfDNA 802 is a double-stranded DNA molecule with an overhang 830. The overhang 830, for instance, includes one or more bases 826 of one ssDNA molecule that extends beyond the corresponding end of the other ssDNA molecule. In some cases, the end motif 824 is defined as the sequence of bases in a single ssDNA within the cfDNA 802 or a sequence of complementary base pairs in both ssDNA within the cfDNA 802. As described herein, the term “endpoint” may refer to at least one of the bases 826 in the end motif 824 and/or overhang 830 of a DNA fragment in a sample.

In various implementations, the cfDNA 802 is obtained from a sample of plasma 832 in the blood 818 of the subject. The plasma 832, for example, includes various DNA fragments 834 including the cfDNA 802. In some cases, the DNA fragments 834 include various types of cfDNA, such as ctDNA and/or cfDNA released from non-cancerous cells.

By sequencing the cfDNA 802, various fragmentomic features may be obtained. These fragmentomic features can be utilized to categorize the cell 804, thereby identifying a tumor heterogeneity of the subject from which the cell 804 was present. In various cases, the fragmentomic features include the presence of at least a portion of the gene 808 in the cfDNA 802. In some cases, the fragmentomic features include the presence of at least a portion of the promotor 810, the enhancer 812, or the variant 814 in the cfDNA 802. In some cases, the fragmentomic features include the presence or sequence of the end motif 824. Other fragmentomic features are described elsewhere herein.

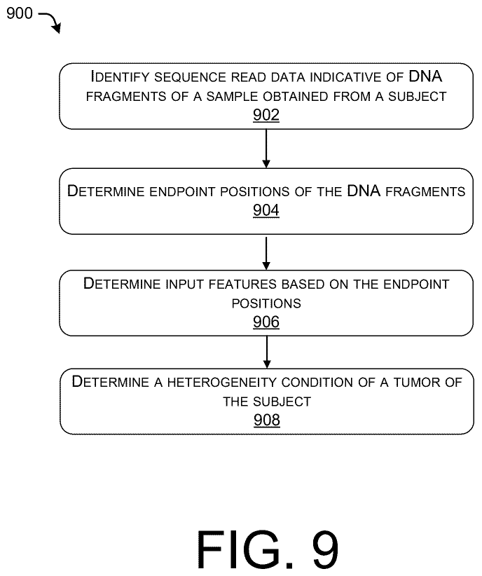

FIG. 9 illustrates an example process 900 for identifying a heterogeneity condition of a subject using fragmentomic data. In various implementations, the process 900 is performed by an entity including at least one processor, at least one computing device, a medical device, the sequencer 112, the preprocessor 116, the data transformer 120, the feature selector 124, the predictive model 128, the report generator 132, the clinical device 136, or any combination thereof.

At 902, the entity identifies sequence read data indicative of DNA fragments of a sample of a subject. For instance, the entity receives a plurality of nucleic acid molecules in a sample of the subject. The sample may include a liquid sample (e.g., a blood sample, a urine sample, a saliva sample, etc.). The nucleic acid molecules, for instance, include genomic DNA from the sample. One or more adapters are ligated onto at least some of the nucleic acid molecules. The ligated molecules are amplified and captured. In various cases, all or a subset of the captured molecules are sequenced to obtain a plurality of sequence reads that represent the sequenced amplified nucleic acid molecules, thereby generating the sequence read data. In particular examples, the sequence read data includes endpoint counts of the DNA fragments at multiple genomic positions within at least one locus of the genome of the sample.

At 904, the entity determines endpoint positions of the DNA fragments with respect to a reference genome. The endpoint positions may include left endpoint positions and/or right endpoint positions of the DNA fragments. In various cases, the entity may determine fragment lengths of the DNA fragments based on, for instance, the left endpoint positions and the right endpoint positions of the DNA fragments. In some examples, the endpoint positions of the DNA fragments are preprocessed. For instance, the endpoint positions of the DNA fragments may be normalized and/or smoothed. In various instances, the endpoint positions of the DNA fragments are transformed into an alternate domain, before or after preprocessing. According to various implementations, the preprocessing may enable identification of features that are indicative of tumor heterogeneity from the endpoint positions of the DNA fragments.

At 906, the entity determines input features based on the endpoint positions of the DNA fragments. The input features in some examples, are indicative of the heterogeneity condition of the subject. In some implementations, the input features may be based on the sequence read data (e.g., the endpoint positions of the DNA fragments), the preprocessed data (e.g., the preprocessed endpoint positions of the DNA fragments), the transformed data (e.g., the preprocessed endpoint positions of the DNA fragments), or any combination thereof. In various examples, the input features may be based on the sequences indicated by the sequence read data. In some cases, the input features may be based on pre-classified data associated with individuals who do or do not have the heterogeneity condition. In various instances, the input features may be based on an image of the endpoint positions and/or the preprocessed endpoint positions. In various instances, the input features may be based on the left endpoint positions and/or the right endpoint positions of the DNA fragments. In various instances, the input features may be based on the fragment lengths of the DNA fragments.

At 908, the entity determines a heterogeneity condition of a tumor of the subject. In some cases, the entity utilized an ML-based classifier to predict whether the subject has the condition. The ML-based classifier, for instance, is pre-trained based on data obtained from a population of individuals that omits the subject. In some cases, the classifier includes at least one of an artificial neural network (ANN), a logistic regression model, a decision tree, a k-nearest neighbor (KNN) model, a support vector machine (SVM), or a naëve Bayes classifier. In some cases, the classifier outputs a likelihood that the subject has a particular heterogeneity condition (or the absence of a particular heterogeneity condition). In some cases, the classifier outputs an indication that the subject has the particular condition (or its absence) when the likelihood exceeds a threshold likelihood.

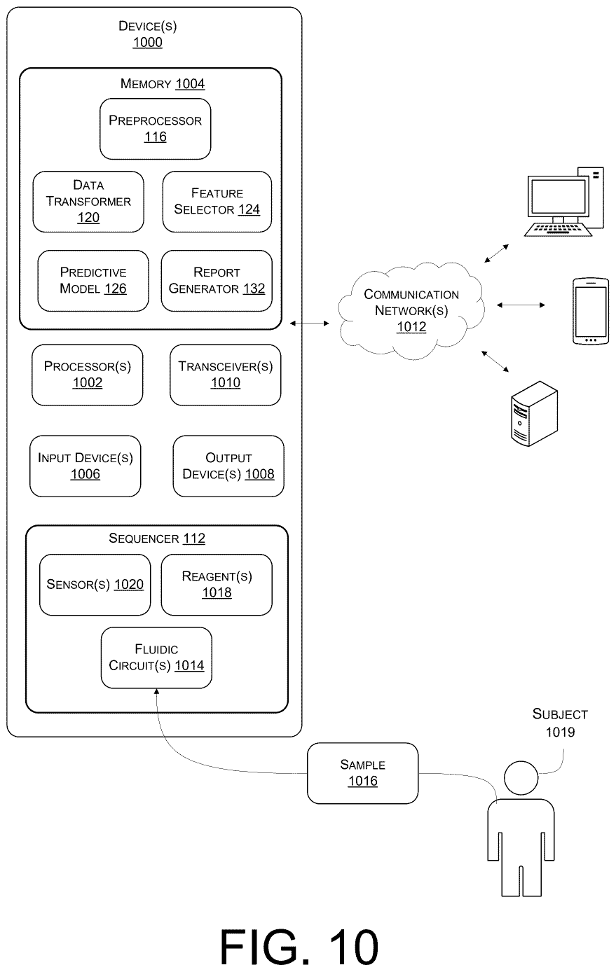

FIG. 10 illustrates one or more devices 1000 configured to perform various operations described herein. The device(s) 1000 include one or more processor(s) 1002. In some implementations, the processor(s) 1002 includes a central processing unit (CPU), a graphics processing unit (GPU), both CPU and GPU, or other processing unit or component known in the art.

The processor(s) 1002 is operably connected to memory 1004. In various implementations, the memory 1004 is volatile (such as random access memory (RAM)), non-volatile (such as read only memory (ROM), flash memory, etc.) or some combination of the two. The memory 1004 stores instructions that, when executed by the processor(s) 1002, causes the processor(s) 1002 to perform various operations. In various examples, the memory 1004 stores methods, threads, processes, applications, objects, modules, any other sort of executable instruction, or a combination thereof. In some cases, the memory 1004 stores files, databases, or a combination thereof. In some examples, the memory 1004 includes, but is not limited to, RAM, ROM, electrically erasable programmable read-only memory (EEPROM), flash memory, or any other memory technology. In some examples, the memory 1004 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 1002. For instance, the memory 1004 stores instructions that, when executed by the processor(s) 1002, causes the processor(s) 1002 to perform operations of the preprocessor 116, data transformer 120, the feature selector 124, the predictive model 128, the report generator 132, or any combination thereof.

The processor(s) 1002 is operably connected to one or more input devices 1006 and one or more output devices 1008. Collectively, the input device(s) 1006 and the output device(s) 1008 function as an interface between at least one user and the device(s) 1000. The input device(s) 1006 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 1008 includes at least one of a display, a speaker, a haptic output device, a printer, or any combination thereof. In various examples, the processor(s) 1002 causes a display among the input device(s) 1006 to visually output various data described herein. In some implementations, the input device(s) 1006 includes one or more touch sensors, the output device(s) 1008 includes a display screen, and the touch sensor(s) are integrated with the display screen.

In various implementations, the processor(s) 1002 is operably connected to one or more transceivers 1010 that transmit and/or receive data over one or more communication networks 1012. For example, the transceiver(s) 1010 includes a network interface card (NIC), a network adapter, a local area network (LAN) adapter, or a physical, virtual, or logical address to connect to the various external devices and/or systems. In various examples, the transceiver(s) 1010 includes any sort of wireless transceivers capable of engaging in wireless communication (e.g., radio frequency (RF) communication). For example, the communication network(s) 1012 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 1010 includes other wireless modems, such as a modem for engaging in WI-FI®, WIGIG®, WIMAX®, BLUETOOTH®, or infrared communication over the communication network(s) 1012.

The device(s) 1000 may further include the sequencer 112. In various implementations, the sequencer 112 includes one or more fluidic circuits 1014 configured to receive a sample 1016 derived from a subject 1019. The sequencer 112, in various cases, may be configured to generate data indicative of one or more sequences of nucleic acid molecules (e.g., DNA and/or RNA) present in the sample 1016. In various cases, the sequencer 112 introduces one or more reagents 1018 to the fluidic circuit(s) 1014 in order to prepare for and perform sequencing of the nucleic acid molecules. Further, the sequencer 112 may include one or more sensors 1020 configured to measure or otherwise detect detection signals from the fluidic circuit(s) 1014, which may be indicative of the sequences of the nucleic acid molecules. According to various implementations, the sensor(s) 1020 may further include one or more ADCs. The sequencer 112, in various cases, outputs sequence read data to the processor(s) 1002 for additional processing.

### Example Clauses

The following clauses provide various non-limiting implementations of the present disclosure:
1. A method, including: providing a plurality of nucleic acid molecules obtained from a sample from a subject, the plurality of nucleic acid molecules including DNA fragments; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, all or a subset of the captured amplified nucleic acid molecules to obtain a plurality of sequence reads that represent the sequenced amplified nucleic acid molecules thereby generating sequence read data; receiving, at one or more processors, the sequence read data for the plurality of sequence reads; generating, by the one or more processors using the sequence read data, endpoint positions of the DNA fragments indicated by the sequence read data with respect to a reference genome; generating, by the one or more processors, input features based on the endpoint positions of the DNA fragments with respect to the reference genome; and identifying, based on the input features and using a classifier executed by the one or more processors, one or more cancer cell populations in a tumor of the subject.
2. The method of clause 1, wherein the sample includes a liquid biopsy sample.
3. The method of clause 1 or 2, wherein the one or more cancer cell populations include one or more distinct cancer cell populations in the tumor.
4. The method of clause 3, wherein the distinct cancer cell populations in the tumor include different cancer cell types and/or different cancer cell subtypes.
5. The method of clause 3 or 4, wherein the one or more distinct cell populations include a first cell population and a second cell population in the tumor, the method further including: determining a first therapeutic agent predicted to treat the first cell population; and determining a second therapeutic agent predicted to treat the second cell population.
6. The method of any of clauses 3-5, the method further including: determining, based on the one or more cancer cell populations, at least one of: a predicted tumor evolution of the subject; a predicted tumor progression of the subject; a predicted metastasis profile of the subject; or a predicted survivability of the subject.
7. The method of any of clauses 3-6, wherein the classifier is a machine learning (ML) classifier, the method further including training the ML classifier to identify attributes based on training data indicative of example DNA fragments identified from example samples of a population, wherein the attributes are indicative of the one or more cancer cell populations, and wherein the input features include instances of the attributes identified via the training of the ML classifier.
8. The method of any of clauses 1-7, further including: determining, based on the sequence read data, a portion of the DNA fragments shed from the one or more cancer cell populations, wherein determining, based on the sequence read data, the endpoint positions of the DNA fragments with respect to the reference genome includes determining endpoint positions of the portion of the DNA fragments shed from the one or more cancer cell populations.
9. A method, including: identifying sequence read data indicating sequences of DNA fragments of a sample obtained from a subject; determining, based on the sequence read data, endpoint positions of the DNA fragments with respect to a reference genome; determining input features based on the endpoint positions of the DNA fragments with respect to the reference genome; and determining, using a classifier and based on the input features, a heterogeneity condition of a tumor of the subject.
10. The method of clause 9, wherein the sequence read data correspond to a single genomic locus.
11. The method of clause 9 or 10, wherein the sequence read data correspond to multiple genomic loci.
12. The method of any of clauses 9-11, further including: determining, based on the sequence read data, a portion of the DNA fragments released from cancer cells of the subject, wherein determining, based on the sequence read data, the endpoint positions of the DNA fragments with respect to the reference genome includes determining endpoint positions of the portion of the DNA fragments released from cancer cells of the subject.
13. The method of any of clauses 9-12, wherein the sample includes a tissue biopsy sample, a liquid biopsy sample, or a normal control.
14. The method of any of clauses 9-13, wherein the sample includes a liquid biopsy sample.
15. The method of clause 14, wherein the liquid biopsy sample includes blood, plasma, cerebrospinal fluid, sputum, stool, urine, lymphatic fluid, or saliva.
16. The method of clause 14 or 15, wherein the liquid biopsy sample includes circulating tumor cells (CTCs).
17. The method of any of clauses 9-16, wherein the sample includes a blood sample.
18. The method of any of clauses 9-17, wherein the sample includes plasma.
19. The method of any of clauses 9-18, wherein the sample includes a tissue biopsy sample.
20. The method of clause 19, wherein the tissue biopsy sample is obtained from a tumor of the subject.
21. The method of clause 20, wherein the tumor includes a primary tumor.
22. The method of clause 20 or 21 wherein the tumor includes a secondary tumor.
23. The method of any of clauses 19-22, wherein the tissue biopsy sample includes an organ and/or differentiated tissue of the subject.
24. The method of any of clauses 9-23, wherein the sample includes at least one of cell-free DNA (cfDNA), circulating tumor DNA (ctDNA), genomic DNA, or hematopoietic stem cells (HSCs).
25. The method of clause 24, wherein the sample includes cfDNA and ctDNA.
26. The method of clause 25, further including: determining, based on an amount of the ctDNA relative to an amount of the cfDNA, a portion of the DNA fragments released from cancer cells of the subject.
27. The method of any of clauses 9-26, wherein the sample includes cell-free DNA (cfDNA) and/or genomic DNA, the cfDNA including ctDNA.
28. The method of clause 27, wherein the DNA fragments include the ctDNA.
29. The method of any of clauses 9-28, further including: receiving the sample.
30. The method of any of clauses 9-29, further including: extracting one or more nucleic acid molecules from the sample.
31. The method of clause 30, wherein the nucleic acid molecules include DNA including the DNA fragments.
32. The method of clause 31, wherein the DNA includes genomic DNA.
33. The method of any of clauses 30-32, wherein the nucleic acid molecules include RNA, the RNA including messenger RNA (mRNA), transfer RNA (tRNA), ribosomal RNA (rRNA), or non-coding RNA, and wherein the input features are further based on the RNA.
34. The method of clause 33, wherein the RNA includes mRNA.
35. The method of clause 33 or 34, wherein the non-coding RNA includes microRNA (miRNA), small interfering RNA (siRNA), Piwi-interacting RNA (piRNA), small Cajal body-specific RNA (scaRNA), long intergenic non-coding RNA (lincRNA), circular RNA (circRNA), enhancer RNA (eRNA), or natural antisense transcripts (NAT).
36. The method of any of clauses 33-35, wherein the non-coding RNA includes miRNA.
37. The method of any of clauses 9-36, further including: ligating one or more adapters onto one or more nucleic acid molecules in the sample, the one or more nucleic acid molecules including the DNA fragments; amplifying the one or more ligated nucleic acid molecules; capturing all or a subset of the amplified nucleic acid molecules; and sequencing, by a sequencer, the captured nucleic acid molecules to obtain a plurality of sequence reads that represent the captured nucleic acid molecules, wherein the sequence read data is indicative of the sequence reads, thereby generating the sequence read data.
38. The method of clause 37, wherein the one or more adapters include at least one of amplification primers, flow cell adapter sequences, substrate adapter sequences, or sample index sequences.
39. The method of clause 37 or 38, wherein the captured nucleic acid molecules are captured from the amplified nucleic acid molecules by hybridization to one or more bait molecules.
40. The method of clause 39, wherein the one or more bait molecules include one or more additional nucleic acid molecules, each of the one or more additional nucleic acid molecules including a region that is complementary to a region of a captured nucleic acid molecule.
41. The method of any of clauses 38-40, wherein amplifying the one or more ligated nucleic acid molecules includes performing a polymerase chain reaction (PCR) amplification technique, a non-PCR amplification technique, or an isothermal amplification technique.
42. The method of any of clauses 38-41, wherein sequencing the captured nucleic acid molecules includes use of a massively parallel sequencing (MPS) technique, whole genome sequencing (WGS), whole exome sequencing, targeted sequencing, direct sequencing, or Sanger sequencing.
43. The method of any of clauses 38-42, wherein sequencing the captured nucleic acid molecules includes next generation sequencing (NGS).
44. The method of any of clauses 38-43, wherein sequencing the captured nucleic acid molecules is performed by a next generation sequencer.
45. The method of any of clauses 38-44, wherein sequencing the captured nucleic acid molecules includes sequencing-by-synthesis or nanopore sequencing.
46. The method of any of clauses 9-45, further including: generating ligated molecules by ligating adapters onto nucleic acid molecules of the sample, the nucleic acid molecules including the DNA fragments; generating amplified ligated molecules by amplifying the ligated molecules; generating, using the amplified ligated molecules, detection signals; detecting, by at least one sensor, the detection signals; and generating the sequence read data based on the detection signals.
47. The method of clause 46, wherein the detection signals include electrical signals and/or optical signals.
48. The method of clause 46 or 47, wherein generating, using the amplified ligated molecules, the detection signals includes simultaneously: synthesizing, by a polymerase using fluorescently tagged nucleotide triphosphates (NTPs), a synthesized nucleic acid molecule based on one of the amplified ligated molecules, and wherein detecting, by the at least one sensor, the detection signals include: detecting, by at least one optical sensor, optical signals emitted by the fluorescently tagged NTPs upon binding to the synthesized nucleic acid molecule, the optical signals being indicative of at least one sequence of the DNA fragments.
49. The method of any of clauses 46-48, wherein generating, using the amplified ligated molecules, the detection signals include simultaneously: directing the amplified ligated molecules through a nanopore extending from a first space to a second space through a substrate, and wherein detecting, by the at least one sensor, the detection signals include: detecting, by sensors disposed in the first space and the second space, an electrical signal over time, the electrical signal being indicative of at least one sequence of the DNA fragments.
50. The method of any of clauses 9-49, wherein the subject is human.
51. The method of any of clauses 9-50, wherein the subject has a disease or a suspected disease.
52. The method of any of clauses 9-51, wherein the subject lacks any apparent disease or other pathological condition.
53. The method of any of clauses 9-52, wherein the subject has a high risk of a cancer.
54. The method of any of clauses 9-53, wherein the subject has a family history of a cancer.
55. The method of any of clauses 9-54, wherein the subject has symptoms associated with a cancer.
56. The method of any of clauses 9-55, wherein the subject has been diagnosed with a cancer.
57. The method of any of clauses 9-56, wherein the subject has a cancer.
58. The method of clause 57, wherein the cancer is adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof.
59. The method of clause 57, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
60. The method of any of clauses 9-59, wherein the endpoint positions of the DNA fragments include multiple genomic positions with respect to the reference genome.
61. The method of any of clauses 9-60, wherein the endpoint positions include left endpoint positions and/or right endpoint positions.
62. The method of clause 61, wherein the DNA fragments extend between the left endpoint positions and the right endpoint positions.
63. The method of any of clauses 9-62, wherein determining the endpoint positions of the DNA fragments includes: aligning the sequences of the DNA fragments to a sequence of the reference genome; and determining the endpoint positions of the DNA fragments aligned with respect to the reference genome.
64. The method of clause 63, wherein aligning the sequences of the DNA fragments to a sequence of the reference genome includes: identifying a quantity and/or presence of variants present in the DNA fragments in the sample.
65. The method of any of clauses 9-64, wherein determining the endpoint positions of the DNA fragments includes determining endpoint positions of the DNA fragments with respect to the reference genome within genomic regions indicated by the sequence read data.
66. The method of clause 65, further including: determining the genomic regions based on a comparison between the sequence read data and reference sequence read data associated with samples corresponding to a plurality of individuals that lack the heterogeneity condition.
67. The method of clause 65 or 66, wherein the genomic region includes at least one of ABL1, ACVR1B, AKT1, AKT2, AKT3, ALDH2, ALK, ALOX12B, AMER1, APC, APOE, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BNIP3, BRAF, BRCA1, BRCA2, BRD4 , BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CK8, CK18, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), ENO1, EP300, EPHA3, EPHB1, EPHB4, ERBB2, ERBB3, ERBB4, ERCC4, ERG, ERRFI1, ESR1, ETV1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXA1, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MRPL44, MSH2, MSH3, MSH6, MST1R, MT-CO1, MT-CO3, MT-ND1, MTAP, MTOR, MUTYH, MYB, MYC, MYCL, MYCN, MYD88, NBN, NDUFV2, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PGK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC16A3, SLC34A2, SMAD2, SMAD4, SMARCA4, SMARCB1, SMO, SNAIL1, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TWIST1, TYRO3, U2AF1, VEGFA, VHL, WHSC1, WHSC1L1, WT1, XPO1, XRCC2, ZNF217, ZNF703, ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB.
68. The method of any of clauses 9-67, wherein determining the input features based on the endpoint positions of the DNA fragments with respect to the reference genome is further based on lengths of the DNA fragments in the sample.
69. The method of any of clauses 9-68, wherein determining the input features based on the endpoint positions of the DNA fragments is further based on local read depths of the DNA fragments in the sample at multiple genomic positions.
70. The method of any of clauses 9-69, wherein determining the input features based on the endpoint positions of the DNA fragments is further based on an endpoint density of the DNA fragments in the sample at multiple genomic positions.
71. The method of any of clauses 9-70, wherein determining the input features based on the endpoint positions of the DNA fragments is further based on a read depth depletion of the DNA fragments in the sample at multiple genomic positions.
72. The method of any of clauses 9-71, wherein determining the input features based on the endpoint positions of the DNA fragments is further based on a gene body depletion of the DNA fragments in the sample at multiple genomic positions.
73. The method of any of clauses 9-72, wherein generating the input features of the sample further includes: determining, based on the sequence read data, a copy number state, and wherein the input features further include the copy number state.
74. The method of clause 73, wherein determining, based on the sequence read data, the copy number state includes: generating, based on the sequence read data, a major allele coverage ratio and a minor allele coverage ratio; segmenting one or more nucleic acid sequences associated with the sequence read data into segments; generating copy number grid model input features including: a sum of the major allele coverage ratio and the minor allele coverage ratio; and a difference of the major allele coverage ratio and the minor allele coverage ratio; fitting copy number grid models including allowed copy number states to the copy number grid model input features; selecting a copy number grid model among the copy number grid models; and assigning the copy number state for at least a portion of the one or more nucleic acid sequences based on the selected copy number grid model.
75. The method of any of clauses 9-74, wherein generating the input features further includes: identifying an image of the sample; and determining, by analyzing the image, a visual characteristic of the sample.
76. The method of any of clauses 9-75, wherein input features further include a histological characteristic or an immunohistological characteristic of the sample.
77. The method of any of clauses 9-76, wherein the input features further include a presence and/or type of one or more variants in the sample.
78. The method of any of clauses 9-77, wherein determining the input features is further based on at least one of: at least one end motif of the DNA fragments; at least one length of the DNA fragments; at least one relative read depth of the DNA fragments; or one or more variants in the DNA fragments.
79. The method of any of clauses 9-78, further including: determining, based on the sequence read data, a distribution of the DNA fragments in the sample, wherein the input data is further based on the distribution of the DNA fragments in the sample.
80. The method of any of clauses 9-79, further including: generating, based on the endpoint positions of the DNA fragments, images representative of the endpoint positions of the DNA fragments.
81. The method of clause 80, wherein the images are representative of genomic regions indicated by the sequence read data.
82. The method of clause 80 or 81, wherein the images representative of the endpoint positions of the DNA fragments include at least one of: a left endpoint position of each of the fragments; a right endpoint position of each of the fragments; and a length of each of the fragments.
83. The method of any of clauses 80-82, wherein the images representative of the endpoint positions of the DNA fragments include a plurality of pixel intensities corresponding to a distribution of the DNA fragments.
84. The method of any of clauses 80-83, wherein the input features are determined based on the images representative of the endpoint positions of the DNA fragments.
85. The method of any of clauses 9-84, wherein the classifier includes a machine learning (ML) classifier.
86. The method of clause 85, wherein the ML classifier includes at least one of a: an artificial neural network (ANN); a logistic regression model; a random forest model, a decision tree; a k-nearest neighbor (KNN) model; a support vector machine (SVM); or a naëve Bayes classifier.
87. The method of clause 85 or 86, further including training the ML classifier based on training data indicative of example DNA fragments identified from example samples of a population.
88. The method of clause 87, wherein the population omits the subject.
89. The method of clause 87 or 88, wherein training the ML classifier is based on supervised machine learning, the training data including labels indicating heterogeneity conditions associated with the example samples.
90. The method of clause 89, wherein the ML classifier is trained to identify attributes, within the training data, that are predictive of the heterogeneity conditions, and wherein the input features include instances of the attributes identified via the training of the ML classifier.
91. The method of clause 89 or 90, wherein training the ML classifier is based on unsupervised machine learning, and wherein training of the ML classifier includes identifying a plurality of clusters of the training data.
92. The method of clause 91, further including: identifying at least one cluster, of the plurality of clusters, associated with one or more example samples associated with the heterogeneity condition, wherein the input features are attributes associated with the at least one cluster.
93. The method of any of clauses 9-92, wherein the input features are determined by: generating transformed data by converting, using a transform, the sequence read data from a spatial domain into an alternative domain; and generating the input features based on the transformed data.
94. The method of clause 93, wherein the alternative domain is a frequency domain.
95. The method of clause 93 or 94, wherein the alternative domain is a wavelet domain.
96. The method of any of clauses 93-95, wherein the transform includes at least one of a Fourier transform, a short-time Fourier transform (STFT), a discrete Fourier transform (DFT), a fast Fourier transform (FFT), a Hartley transform, a Laplace transform, a Mellin transform, or a Wavelet transform.
97. The method of any of clauses 93-96, further including applying at least one filter to the transformed data, the at least one filter including one or more of a high-pass filter, a low-pass filter, a Butterworth filter, a Chebyshev filter, a finite impulse response (FIR) filter, or an infinite impulse response (IIR) filter.
98. The method of clause 97, wherein applying the at least one filter to the transformed data includes multiplying the at least one filter with the transformed data.
99. The method of any of clauses 93-98, wherein: the classifier includes an ML classifier, a training data set indicates, in the spatial domain, example endpoint positions of example DNA fragments identified from example samples of a population, the ML classifier is trained based on translated training data expressed in the alternative domain, generated by applying the transform to the training data set, to identify attributes that are predictive of the subjects having the heterogeneity condition, and the input features are instances of the attributes identified via the training of the ML classifier.
100. The method of any of clauses 93-99, wherein generating the input features based on the transformed data includes: generating a digital image based on the transformed data; and extracting the input features from the digital image using a convolutional neural network (CNN).
101. The method of clause 100, wherein: the CNN includes a plurality of layers, a layer, of the plurality of layers, includes a kernel associated with one or more parameters, and extracting the input features from the digital image includes generating an output image by at least one of convolving or cross-correlating the kernel with an input image based on the digital image.
102. The method of clause 101, further including training the CNN based on training data including example input images and corresponding example outputs, wherein training the CNN includes adjusting parameters of one or more of the plurality of layers to minimize a loss between the example outputs and outputs generated by the CNN based on the example input images.
103. The method of clause 102, wherein the training data is pre-classified data generated by: identifying training sequence read data associated with example samples of a population; generating the training data by transforming the training sequence read data into the alternative domain using the transform; and labeling the training data with labels indicative of conditions of example subjects in the population.
104. The method of any of clauses 9-103, further including: determining a frequency distribution of endpoint counts of the DNA fragments indicated by the sequence read data; generating a normalized frequency distribution by normalizing the frequency distribution; generating a smoothed frequency distribution by smoothing the normalized frequency distribution; and generating scaled endpoint data, representative of the frequency distribution, by scaling the smoothed frequency distribution based on a plurality of control samples.
105. The method of clause 104, wherein generating the normalized frequency distribution includes normalizing the frequency distribution based on a mean of the frequency distribution of the endpoint counts.
106. The method of clause 104 or 105, wherein generating the smoothed frequency distribution includes determining a metric over a window of genomic positions centered on an example genomic position of the normalized frequency distribution, and assigning the metric to the example genomic position.
107. The method of clause 106, wherein the metric includes an average endpoint count, a weighted average endpoint count, a median endpoint count, a kernel function, or a filter.
108. The method of any of clauses 104-107, wherein generating the scaled endpoint data includes: receiving control sequence read data associated with a plurality of control subjects; and determining a distance metric by comparing the smoothed frequency distribution to a control frequency distribution indicated by the control sequence read data.
109. The method of clause 108, wherein the distance metric is based on the scaled frequency distribution and at least one of the control frequency distribution, a mean of the control frequency distribution, or a standard deviation of the control frequency distribution.
110. The method of clause 109, wherein generating the scaled endpoint data includes scaling the smoothed frequency distribution into a z-score space based on the at least one of the control frequency distribution, the mean of the control frequency distribution, or the standard deviation of the control frequency distribution.
111. The method of any of clauses 9-110, wherein the heterogeneity condition includes a metric indicative of one or more cell populations in the tumor of the subject.
112. The method of any of clauses 9-111, wherein the heterogeneity condition of the tumor includes a mutational profile, alteration-level clonality, or a cell type profile of the tumor.
113. The method of any of clauses 9-112, wherein the heterogeneity condition indicates distinct cell populations in the tumor.
114. The method of clause 113, wherein the distinct cell populations in the tumor include different cancer cell types and/or different cancer cell subtypes.
115. The method of clause 113 or 114, wherein the distinct cell populations in the tumor have different responses to one or more treatments.
116. The method of any of clauses 9-115, further including: determining, based on the heterogeneity condition, at least one of: a tumor evolution of the subject; a predicted tumor evolution of the subject; a predicted tumor progression of the subject; a predicted pathologic condition of the subject; a predicted pathologic condition subtype of the subject; a predicted metastasis profile of the subject; a predicted survivability of the subject; a predicted symptom of the subject; a predicted effective therapy to treat the predicted pathologic condition of the subject; a predicted resistance of the subject to a treatment of the predicted pathologic condition; a general health of the subject; a genomic age of the subject; a risk of the subject developing the predicted pathologic condition; a predicted stage of the predicted pathologic condition of the subject; a predicted grade of the predicted pathologic condition of the subject; or a predicted Eastern Cooperative Oncology Group (ECOG) performance status of the subject.
117. The method of any of clauses 9-116, wherein the heterogeneity condition indicates an evolution of the tumor of the subject, a predicted progression of the tumor of the subject, or at least one predicted effective therapy for treatment of the tumor of the subject.
118. The method of any of clauses 9-117, wherein the sample includes hematopoietic stem cells (HSCs) and the heterogeneity condition indicates a risk of the subject developing a blood cancer.
119. The method of clause 118, wherein the blood cancer includes a myelodysplastic syndrome, acute myeloid leukemia, acute lymphoblastic leukemia, chronic lymphocytic leukemia, chronic myeloid leukemia, leukemia, Hodgkin lymphoma, non-Hodgkin lymphoma, lymphoma, multiple myeloma, or a myeloproliferative neoplasm.
120. The method of any of clauses 9-119, wherein the heterogeneity condition indicates a presence of germline variants in cells of the tumor.
121. The method of any of clauses 9-120, wherein the heterogeneity condition indicates a presence of somatic variants in cells of the tumor.
122. The method of any of clauses 9-121, further including: generating, based on the heterogeneity condition, a genomic profile of the subject.
123. The method of clause 122, wherein the genomic profile includes results from at least one of: a histological study, whole transcriptome sequencing, cfRNA sequencing, a comprehensive genomic profiling test; a whole genome sequencing (WGS) test; a whole exome sequencing (WES) test; a gene expression profiling test; a cancer hotspot panel test; a DNA methylation test; a DNA fragmentation test; or an RNA fragmentation test, a microsatellite instability (MSI) test, a tumor mutational burden (TMB) test, or a viral status test.
124. The method of clause 122 or 123, wherein the genomic profile of the subject includes: results from a nucleic acid sequencing-based test.
125. The method of any of clauses 122-124, further including: generating, based on the heterogeneity condition and/or genomic profile, a therapy for the subject.
126. The method of clause 125, wherein the therapy includes drug therapy, radiation therapy, a targeted therapy, vaccine therapy, stem cell transplantation, blood transfusion, physical therapy, psychiatric therapy, or surgery.
127. The method of clause 126, wherein the drug therapy includes chemotherapy.
128. The method of clause 126 or 127, wherein the targeted therapy includes immunotherapy or genetic therapy.
129. The method of any of clauses 125-128, wherein the therapy includes a dosage of one or more therapeutic agents predicted to treat a condition of the subject.
130. The method of any of clauses 125-129, wherein the heterogeneity condition indicates one or more distinct cell populations in the tumor, and wherein the therapy includes one or more therapeutic agents predicted to treat each of the one or more distinct cell populations in the tumor.
131. The method of any of clauses 125-130, wherein the heterogeneity condition indicates a first cell population and a second cell population in the tumor, the method further including: determining a first therapeutic agent predicted to treat the first cell population; and determining a second therapeutic agent predicted to treat the second cell population.
132. The method of clause 131, further including: administering the first therapeutic agent and the second therapeutic agent to the subject.
133. The method of any of clauses 125-132, wherein the heterogeneity condition indicates a first cell population and a second cell population in the tumor, the method further including: determining that the first cell population is responsive to a therapeutic agent; and determining that the second cell population is not responsive to the therapeutic agent.
134. The method of any of clauses 122-133, further including: selecting, based on the heterogeneity condition and/or genomic profile, one or more therapeutic agents for administration to the subject.
135. The method of clause 134, further including: administering the one or more therapeutic agents to the subject.
136. The method of any of clauses 122-135, further including: determining, based on the heterogeneity condition and/or genomic profile, whether the subject is eligible for a clinical trial.
137. The method of any of clauses 9-136, further including determining, based on the heterogeneity condition whether to perform a follow-up diagnostic test.
138. The method of clause 137, further including performing the follow-up diagnostic test.
139. The method of clause 137 or 138, wherein the follow-up diagnostic test includes a physical exam, biopsy, sequencing-based test, diagnostic imaging, histological study, or viral status test.
140. The method of clause 139, wherein the biopsy includes obtaining a tissue biopsy sample of a tumor.
141. The method of clause 140, wherein the tumor is a primary tumor.
142. The method of clause 140 or 141, wherein the tumor is a secondary tumor.
143. The method of any of clauses 139-142, wherein the sequencing-based test includes whole transcriptome sequencing, cfRNA sequencing, whole exome sequencing, whole genome sequencing, a cancer hotspot panel test, a DNA methylation test, a DNA fragmentation test, an RNA fragmentation test, a microsatellite instability (MSI) test, or a tumor mutational burden (TMB) test.
144. The method of any of clauses 139-143, wherein the diagnostic imaging includes magnetic resonance imaging, computed tomography scan, ultrasound, X-ray, mammogram, positron emission tomography, bone scintigraphy, myelography, virtual colonoscopy, echocardiography, radiography, nuclear medicine, fluoroscopy, or single-photon emission computed tomography.
145. The method of any of clauses 137-144, wherein the follow-up diagnostic test includes at least one of: whole transcriptome sequencing; cfRNA sequencing; or an RNA fragmentation test.
146. The method of any of clauses 9-145, further including determining, based on the heterogeneity condition whether the subject is eligible for a clinical trial.
147. The method of clause 146, wherein determining, based on the heterogeneity condition, whether the subject is eligible for the clinical trial includes determining that the subject matches inclusion criteria for the clinical trial.
148. The method of clause 147, wherein the inclusion criteria include criteria for age, gender, disease stage, and previous treatments.
149. The method of any of clauses 146-148, wherein determining, based on the heterogeneity condition, whether the subject is eligible for the clinical trial includes determining that the subject is taking one or more specific medications.
150. The method of any of clauses 146-149, wherein determining, based on the heterogeneity condition, whether the subject is eligible for the clinical trial includes determining that the subject is not taking any medications.
151. The method of any of clauses 146-150, wherein the subject is not eligible for a clinical trial.
152. The method of any of clauses 9-151, further including: generating a report based on the heterogeneity condition; and outputting the report.
153. The method of clause 152, wherein outputting the report includes: transmitting data indicating the report to an external device.
154. The method of clause 153, wherein the external device is associated with the subject and/or a healthcare provider.
155. The method of clause 153 or 154, wherein the data is transmitted over one or more communication networks.
156. The method of any of clauses 153-155, wherein the data is transmitted over a peer-to-peer connection.
157. The method of any of clauses 153-156, wherein outputting the report includes: visually presenting, by a display, the report.
158. The method of any of clauses 153-157, wherein the report indicates the heterogeneity condition.
159. A system, including: at least one processor; and memory storing instructions that, when executed by the at least one processor, cause the at least one processor to perform operations including: identifying sequence read data indicating sequences of DNA fragments of a sample obtained from a subject; determining, based on the sequence read data, endpoint positions of the DNA fragments with respect to a reference genome; determining input features based on the endpoint positions of the DNA fragments with respect to the reference genome; and determining, using a classifier and based on the input features, a heterogeneity condition of a tumor of the subject.
160. The system of clause 159, further including: a sequencer configured to generate the sequence read data by sequencing a plurality of nucleic acid molecules in the sample.
161. The system of clause 159 or 160, further including: a transceiver configured to transmit data indicating the heterogeneity condition of the tumor of the subject.
162. The system of any of clauses 159-161, further including: an output device configured to output an indication of the heterogeneity condition of the tumor of the subject.
163. A non-transitory computer readable medium storing instructions for performing operations including: identifying sequence read data indicating sequences of DNA fragments of a sample obtained from a subject; determining, based on the sequence read data, endpoint positions of the DNA fragments with respect to a reference genome; determining input features based on the endpoint positions of the DNA fragments with respect to the reference genome; and determining, using a classifier and based on the input features, a heterogeneity condition of a tumor of the subject.
164. A method of identifying an individual having a tumor with a heterogeneity condition, the method including detecting in a sample from the individual having the tumor: a predetermined pattern of endpoint positions of DNA fragments obtained from the sample of the individual, wherein detection of predetermined pattern of endpoint positions of the DNA fragments identifies the individual as one who may have the tumor with the heterogeneity condition.
165. A method of treating or delaying progression of cancer in an individual in need thereof, including: acquiring knowledge of: endpoint positions of DNA fragments obtained from a sample of the individual; determining, based on the endpoint positions of the DNA fragments, a heterogeneity condition of a tumor of the individual; selecting a treatment based on the endpoint positions of the DNA fragments; and administering to the individual an effective amount of the treatment.

### Conclusion

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, the practice of the present disclosure can employ conventional techniques of immunology, molecular biology, microbiology, cell biology and recombinant DNA. These methods are described in the following publications. See, e.g., Green and Sambrook, Molecular Cloning: A Laboratory Manual, 4nd Edition (2012); F. M. Ausubel, et al. eds., Current Protocols in Molecular Biology, (2003); the series Methods In Enzymology (Academic Press, Inc.); Behlke, et al., Polymerase Chain Reaction: Theory and Technology (2019); Greenfield, ed. Antibodies, A Laboratory Manual, Second Edition (2014); and Capes-Davis and R. I. Freshney, eds. Freshney's Culture of Animal Cells 8th Edition (2021).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A method, comprising:
providing a plurality of nucleic acid molecules obtained from a sample from a subject, the plurality of nucleic acid molecules comprising DNA fragments;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, all or a subset of the captured amplified nucleic acid molecules to obtain a plurality of sequence reads that represent the sequenced amplified nucleic acid molecules thereby generating sequence read data;
receiving, at one or more processors, the sequence read data for the plurality of sequence reads;
generating, by the one or more processors using the sequence read data, endpoint positions of the DNA fragments indicated by the sequence read data with respect to a reference genome;
generating, by the one or more processors, input features based on the endpoint positions of the DNA fragments with respect to the reference genome; and
identifying, based on the input features and using a classifier executed by the one or more processors, one or more cancer cell populations in a tumor of the subject.

2. The method of claim 1, wherein the one or more cancer cell populations comprise one or more distinct cancer cell populations in the tumor, the distinct cancer cell populations in the tumor comprising different cancer cell types and/or different cancer cell subtypes.

3. The method of claim 2, wherein the one or more distinct cell populations comprise a first cell population and a second cell population in the tumor, the method further comprising:
determining a first therapeutic agent predicted to treat the first cell population; and
determining a second therapeutic agent predicted to treat the second cell population.

4. The method of claim 1, the method further comprising: determining, based on the one or more cancer cell populations, at least one of:
a predicted tumor evolution of the subject;
a predicted tumor progression of the subject;
a predicted metastasis profile of the subject; or
a predicted survivability of the subject.

5. A method, comprising:
identifying sequence read data indicating sequences of DNA fragments of a sample obtained from a subject;
determining, based on the sequence read data, endpoint positions of the DNA fragments with respect to a reference genome;
determining input features based on the endpoint positions of the DNA fragments with respect to the reference genome; and
determining, using a classifier and based on the input features, a heterogeneity condition of a tumor of the subject.

6. The method of claim 5, further comprising:
determining, based on the sequence read data, a portion of the DNA fragments released from cancer cells of the subject,
wherein determining, based on the sequence read data, the endpoint positions of the DNA fragments with respect to the reference genome comprises determining endpoint positions of the portion of the DNA fragments released from cancer cells of the subject.

7. The method of claim 5, wherein the sample comprises at least one of cell-free DNA (cfDNA), circulating tumor DNA (ctDNA), genomic DNA, or hematopoietic stem cells (HSCs).

8. The method of claim 5, wherein the subject has a high risk of a cancer or has symptoms associated with cancer.

9. The method of claim 5, wherein determining the endpoint positions of the DNA fragments comprises:
determining genomic regions based on a comparison between the sequence read data and reference sequence read data associated with samples corresponding to a plurality of individuals that lack the heterogeneity condition; and
determining endpoint positions of the DNA fragments with respect to the reference genome within the genomic regions indicated by the sequence read data.

10. The method of claim 5, wherein input features further comprise a histological characteristic or an immunohistological characteristic of the sample.

11. The method of claim 5, wherein determining the input features is further based on at least one of:
at least one end motif of the DNA fragments;
at least one length of the DNA fragments;
at least one relative read depth of the DNA fragments; or
one or more variants in the DNA fragments.

12. The method of claim 5, wherein the classifier comprises a machine learning (ML) classifier, the method further comprising training the ML classifier to identify attributes based on training data indicative of example DNA fragments identified from example samples of a population,
wherein the attributes are indicative of the heterogeneity condition, and wherein the input features comprise instances of the attributes identified via the training of the ML classifier.

13. The method of claim 5, further comprising:
determining a frequency distribution of endpoint counts of the DNA fragments indicated by the sequence read data;
generating a normalized frequency distribution by normalizing the frequency distribution;
generating a smoothed frequency distribution by smoothing the normalized frequency distribution; and
generating scaled endpoint data, representative of the frequency distribution, by scaling the smoothed frequency distribution based on a plurality of control samples.

14. The method of claim 5, wherein the heterogeneity condition of the tumor comprises a mutational profile, alteration-level clonality, or a cell type profile of the tumor.

15. The method of claim 5, wherein the heterogeneity condition indicates an evolution of the tumor of the subject, a predicted progression of the tumor of the subject, or at least one predicted effective therapy for treatment of the tumor of the subject.

16. The method of claim 5, wherein the heterogeneity condition indicates a presence of germline variants in cells of the tumor and/or a presence of somatic variants in cells of the tumor.

17. The method of claim 5, further comprising:
generating, based on the heterogeneity condition, a therapy for the subject, the therapy comprising a dosage of one or more therapeutic agents predicted to treat a condition of the subject.

18. The method of claim 5, wherein the heterogeneity condition indicates a first cell population and a second cell population in the tumor, the method further comprising:
determining that the first cell population is responsive to a therapeutic agent; and
determining that the second cell population is not responsive to the therapeutic agent.

19. The method of claim 5, further comprising:
generating a report based on the heterogeneity condition; and
outputting the report.

20. A system, comprising:
at least one processor; and
memory storing instructions that, when executed by the at least one processor, cause the at least one processor to perform operations comprising:
identifying sequence read data indicating sequences of DNA fragments of a sample obtained from a subject;
determining, based on the sequence read data, endpoint positions of the DNA fragments with respect to a reference genome;
determining input features based on the endpoint positions of the DNA fragments with respect to the reference genome; and
determining, using a classifier and based on the input features, a heterogeneity condition of a tumor of the subject.
